SEQUENCE LISTING (7 sequences)

SEQ ID NO: 1 - protein, 125 aa, Ixodes scapularis
MGLTGTMLVLVSLAFFGSAAAHNCQNGTRPASEQDREGCDYYCWNAETKSWDQFFFGNGEKCFYNSGDHGTCQNGECHLTNNSGGPNETDDYTPAPTEKPKQKKKKTKKTKKPKRKSKKDQEKNL

SEQ ID NO: 2 - protein, 21 aa, Ixodes scapularis
HNCQNGTRPASEQDREGCDYY

SEQ ID NO: 3 - protein, 20 aa, Ixodes scapularis
GCDYYCWNAETKSWDQFFFG

SEQ ID NO: 4 - protein, 20 aa, Ixodes scapularis
QFFFGNGEKCFYNSGDHGTC

SEQ ID NO: 5 - protein, 24 aa, Ixodes scapularis
DHGTCQNGECHLTNNSGGPNETDD

SEQ ID NO: 6 - protein, 20 aa, Ixodes scapularis
PNETDDYTPAPTEKPKQKKK

SEQ ID NO: 7 - protein, 25 aa, Ixodes scapularis
KQKKKKTKKTKKPKRKSKKDQEKNL

(19) United States
(12) Patent Application Publication
(10) Pub. No.: US 2025/0134972 A1
(43) Pub. Date: May 1, 2025

(54) COMPOSITIONS AND METHODS FOR GENERATING TICK IMMUNITY

(71) Applicant: Yale University, New Haven, CT (US)

(72) Inventor: Erol FIKRIG, New Haven, CT (US)

(21) Appl. No.: 18/684,124

(22) PCT Filed: Aug. 18, 2022
(86) PCT No.: PCT/US2022/075112

§ 371 Date: Feb. 15, 2024

Related U.S. Application Data
(60) Provisional application No. 63/234,476, filed on Aug. 18, 2021.

(51) Int. Cl. A61K 39/00; A61K 9/00; A61P 33/14

(52) U.S. Cl. CPC A61K 39/0003; A61K 9/0019; A61P 33/14; A61K 2039/54; A61K 2039/55505; A61K 2039/55566

(57) ABSTRACT

Described herein is a method of generating tick immunity in a subject. The method includes administering to the subject in need thereof a therapeutically effective amount of at least one tick-salivary protein. Also described herein is a composition for generating tick immunity in a subject. In certain embodiments, the composition includes a therapeutically effective amount of at least one tick-salivary protein.

### CROSS-REFERENCE TO RELATED APPLICATIONS

The present application claims priority under 35 U.S.C. § 119(e) to U.S. Provisional Patent Application No. 63/234,476, filed Aug. 18, 2021, which is incorporated herein by reference in its entirety.

### STATEMENT REGARDING FEDERALLY SPONSORED RESEARCH

This invention was made with government support under AI152206, AI126033 and AI138949 awarded by National Institutes of Health. The government has certain rights in the invention.

### SEQUENCE LISTING

The ASCII text file named“047162-7338W01(01765)_Seq Listing” created on Aug. 17, 2022, comprising 6.97 Kbytes, is hereby incorporated by reference in its entirety.

### BACKGROUND

Tick-borne diseases are currently increasing in North America and Europe. The black-legged tick, Ixodes scapularis, transmits diverse pathogens, including Borrelia burgdorferi (the Lyme disease agent), Babesia microti, Anaplasma phagocytophilum, Borrelia miyamotoi, and Powassan virus, among other infectious agents. As one example, Lyme disease, the most common I. scapularis-borne human illness in the U.S., results in almost 40,000 cases reported annually, and the CDC estimates that the real number of infections may be actually 10 times greater. Given the identification of new types of tick-borne pathogens along with their remarkable diversity and generally unsuccessful efforts towards the development of single, pathogen-specific vaccines, the development of a broad anti-tick vaccine strategy is highly desirable. In fact, a possible approach to the prevention of one or more I. scapularis-borne diseases involves developing new methods to detect the ticks early-on and preventing the arthropod from taking a successful blood meal.

The ability of animals to develop acquired resistance to tick bites following repeated exposure to ticks—so-called “acquired tick resistance” or “tick immunity”—was first described by Trager in 1939. Tick immunity is associated with the recruitment of inflammatory cells to the tick bite site, including basophils that degranulate to secrete histamine, thus altering tick feeding.

Naturally acquired tick resistance is generally considered to be associated with host immune responses to tick antigens that are secreted into the bite site, and present in saliva and cement. However, it is unclear whether these antigens are directly involved in the genesis of tick immunity.

There is thus a need in the art for novel compositions and methods for generating tick immunity in individuals in need thereof. The present invention addresses this need.

### SUMMARY

In some aspects, the present invention is directed to the following non-limiting embodiments:

In some embodiments, the present invention is directed to a method of generating tick immunity in a subject.

In some embodiments, the method comprising administering to the subject in need thereof a therapeutically effective amount of at least one tick-salivary protein, wherein the at least one tick-salivary protein comprises at least one protein selected from the group consisting of: Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09.

In some embodiments, the at least one tick-salivary protein comprises Salp14, Salp26A, TSLPI, IsPDIA3, TIX5, P32, and SG27.

In some embodiments, the at least one tick-salivary protein further comprises at least one protein selected from the group consisting of Salp10, Salp15, Salp25A, Salp25B, Salp25C, Salp25D, P11, Salp16A, Salp17, Salp12, SG10, and SG09.

In some embodiments, the at least one tick-salivary protein comprises Salp14, Salp15, Salp25D, Salp26A, TSLPI, IsPDIA3, TIX5, P32, SG10, and SG27.

In some embodiments, the at least one tick-salivary protein comprises Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09

In some embodiments, the method further comprises administering an adjuvant to the subject.

In some embodiments, the adjuvant is at least one selected from the group consisting of incomplete Freund's adjuvant, alum, addavax (equivalent to MF59), MF59, and AS03.

In some embodiments, the at least one tick-salivary protein is administered by at least one route selected from the group consisting of inhalational, oral, rectal, vaginal, parenteral, intracranial, topical, transdermal, intradermal, subcutaneous, pulmonary, intranasal, buccal, ophthalmic, intrathecal, and intravenous.

In some embodiments, the subject is a mammal.

In some embodiments, the subject is a human.

### Composition

In some aspects, the present invention is directed to a composition. In some embodiments, the composition is a composition for generating tick immunity, such as in a subject in need thereof.

In some embodiments, the composition comprises a therapeutically effective amount of at least one tick-salivary protein, wherein the at least one tick-salivary protein comprises at least one protein selected from the group consisting of: Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09.

In some embodiments, the at least one tick-salivary protein comprises Salp14, Salp26A, TSLPI, IsPDIA3, TIX5, P32, and SG27.

In some embodiments, the at least one tick-salivary protein further comprises at least one protein selected from the group consisting of Salp10, Salp15, Salp25A, Salp25B, Salp25C, Salp25D, P11, Salp16A, Salp17, Salp12, SG10, and SG09.

In some embodiments, the at least one tick-salivary protein comprises Salp14, Salp15, Salp25D, Salp26A, TSLPI, IsPDIA3, TIX5, P32, SG10, and SG27.

In some embodiments, the at least one tick-salivary protein comprises Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09.

In some embodiments, the composition further comprises an adjuvant.

In some embodiments, the adjuvant is at least one selected from the group consisting of incomplete Freund's adjuvant, alum, addavax (equivalent to MF59), MF59, and AS03.

In some embodiments, the composition further comprises at least one pharmaceutically acceptable carrier.

In some embodiments, the composition is formulated for administration by at least one route selected from the group consisting of inhalational, oral, rectal, vaginal, parenteral, intracranial, topical, transdermal, pulmonary, intranasal, buccal, ophthalmic, intrathecal, and intravenous.

In some embodiments, the composition is a pharmaceutical composition for generating tick immunity in a subject.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1 depicts antibody responses to specific antigens in guinea pigs according to some embodiments. Specifically, FIG. 1 depicts the ELISA result of guinea pig sera using specific recombinant proteins corresponding to the mRNAs in 19ISP. Sera was isolated from animals after vaccination. ELISA was performed with 19ISP antigens using serum dilutions of 1:500, 1:5,000, and 1:50,000. Control sera were tested at 1:500 and 1:5,000 dilutions. Antibodies were detected against 10 of the tested proteins—Salp14, Salp15, Salp25D, Salp26A, TSLPI, IsPDIA3, TIX5, P32, SG10, and SG27, with the OD at 450 nm in the 19ISP-immunized groups being higher than the controls. The data represents mean±SEM of at least 6 values.

FIG. 2 depicts the tick challenge of 19ISP mRNA-LNP immunized guinea pigs according to some embodiments. As depicted in FIG. 2, guinea pigs were immunized with 19ISP or control (IL21) mRNA and 25 I. scapularis nymphs were allowed to engorge on their shaved backs. All animals were monitored for the development of erythema as a cardinal initial sign of acquired tick resistance over a period of six days or until all ticks detached. The images show representative (upper panels) 19ISP-immunized or (lower panels) control animals. As observed in the images, 19ISP-immunized guinea pigs showed significant erythema and nymphs were fed poorly with earlier detachment, in comparison with controls.

FIGS. 3A-3F demonstrate that 19ISP mRNA vaccination elicits protective responses against tick challenge and B. burgdorferi transmission according to some embodiments. As shown in FIGS. 3A-3F, immunized animals (19ISP n=6, IL21 n=5) were challenged with I. scapularis nymphs and assessed for the follows: FIG. 3A, Erythema: calculated as the percent of nymphs showing redness on each animal, each symbol represents one animal challenged with approximately 25 nymphs. FIG. 3B, Tick-detachment: The graph represents the percent of ticks remaining attached at a given time point in all animals of a group. FIG. 3C, Recovery: The graph shows the percent of total ticks recovered from each animal after rejection or detachment. FIG. 3D, Engorgement: Weights of ticks recovered from each animal after rejection or detachment, represented by each symbol. The error bars represent mean±SD and the significance calculated using the Mann-Whitney test. FIG. 3E, B. burgdorferi transmission: qPCR data from skin biopsies from Guinea pigs (immunized with 19ISP, n=16 or Luc-mRNA control, n=13), challenged with B. burgdorferi-infected ticks. Data shows relative levels of B. burgdorferi flaB, normalized with guinea pig actin. The data from three biological replicates shows 6/13 B. burgdorferi infected samples in control versus 0/16 infected samples in the 19ISP group. Error bars show mean±SEM and significance was calculated using Welch's t-test (95% confidence interval). FIG. 3F shows total number of B. burgdorferi culture positive and negative samples in each group, also corroborated by qPCR shown in FIG. 3E.

FIGS. 4A-4B depicts the gene expression analyses by RNAseq according to some embodiments. In FIGS. 4A-4B, RNA-seq analyses were performed using Partek Genomics Flow software. FIG. 4A: Heatmap showing that 125 differentially expressed genes were identified (p<0.05) and fold change greater than or equal to 2.0. 113 genes were upregulated and 12 genes were downregulated in the 19ISP-immunized group as compared to the control group. FIG. 4B: Signaling pathways involved in response to 19ISP-mRNA vaccination were identified by KEGG pathway enrichment analysis. The top immune pathways enriched are T cell receptor, B-cell receptor signaling, chemokine signaling, IL-17 signaling, Natural killer cell-mediated cytotoxicity, Fc epsilon RI mediated signaling, and C type lectin receptor signaling.

FIGS. 5A-5F depict the cytokine expression in 19ISP-immunized guinea pigs according to some embodiments. As shown in these figures, cytokine expression in RNA from PBMCs isolated from 19ISP and control mRNA immunized guinea pigs, 2-weeks after the second boost and stimulated with I. scapularis saliva qRT-PCR shows relative expression calculated using the deltaCq method and normalized with guinea pig gapdh: FIG. 5A, IFNγ. FIG. 5B, TNFα, FIG. 5C, CXCL10. FIG. 5D, IL2, FIG. 5E, IL4. FIG. 5F, IL8. The figures show data from two independent experiments and each circle represents a guinea pig (n=6). The p-value was calculated using unpaired t-test, between saliva stimulated 19ISP and mRNA control and error bars show mean SEM.

FIGS. 6A-6D demonstrate that the 7 mRNAs that elicit the strongest antibody responses (“High titer” in FIG. 6A) in FIG. 1, when administered together (“High” in FIGS. 6B-6C), elicit significant antibody response. FIG. 6A displays the nineteen tick saliva proteins in three groups based on the mRNA antibody response strength as essayed by ELISA. In FIGS. 6B-6C guinea pigs were administered with the combined mRNAs of the saliva proteins of the high titer group, the medium titer group, or the no titer group, and essayed for tick attachment (FIG. 6B), redness score (FIG. 6C), and tick weight (FIG. 6D).

FIGS. 7A-7B demonstrate that various Salp 14 immunization strategies elicit Salp14 IgG responses in guinea pigs. FIG. 7A shows that guinea pigs were immunized intradermally with salp14 mRNA-LNPs or murine (mu)IL-21 mRNA-LNP (control), salp14 DNA or empty plasmid VR2010 (control), Salp14 protein (bolus and sustained) or Ovalbumin (OVA control). Two weeks after the last immunization, sera were collected to assess the humoral immune response and challenged with 25 nymphal I. scapularis ticks. Illustration generated using Biorender.com. FIG. 7B shows that Salp14-specific IgG antibodies were detected by ELISA. The error bars represent mean±SEM of at least 3 values.

FIGS. 8A-8C show comparison of erythematic response generation according to some embodiments. Guinea pigs were challenged with I. scapularis nymphs and monitored for erythema at the bite sites. Graphs show erythema after tick challenge in the animals immunized with FIG. 8A, salp14 DNA (n=3), empty plasmid VR2010 (control) (n=3). FIG. 8B, salp14 mRNA (n=3), murine (mu)IL-21 mRNA-LNP (control) (n=3). FIG. 8C, Salp14 protein bolus (n=3), sustained (n=3) and Ovalbumin (OVA control) (n=3). Immunization with salp14-mRNA elicits robust erythema upon tick challenge. The error bars represent mean±SEM. Statistical significance was determined by two-way ANOVA; In FIGS. 7A-7B, P value <0.0001. In FIG. 8C, P value=0.0068.

FIGS. 9A-9C shows that rapid erythema induced at the bite site of mRNA-salp14 vaccinated animals. As shown in FIG. 9A, guinea pigs were monitored following the tick challenge and redness at the bite site was photographed. Representative animals are shown at 16, 24 and 48 hours post-tick challenge. As shown in FIGS. 9B and 9C, visual inspection was performed to determine the degree of erythema at the site of the tick bite at 24 hours (FIG. 9B) and 48 hours (FIG. 9C). Erythema was scored as: No erythema (−), Light (+), Moderate (++), Strong (+++), and the percentage of the degree of erythema was generated from these data.

FIGS. 10A-10F depicts the tick feeding kinetics on immunized guinea pigs. Guinea pigs (n=3 per group) were immunized with salp14 DNA (FIGS. 10A and 10D), salp14-mRNA (FIGS. 10B and 10E), and Salp14 protein (bolus and sustained) (FIGS. 10 and 10F) and challenged with I. scapularis. Guinea pigs were monitored for evidence of tick rejection and the tick feeding kinetics were monitored for the duration of the experiment (FIGS. 10A-10C). The success of tick feeding was determined by examining engorgement weights of the recovered ticks (FIGS. 10D-10F). Statistical significance was determined (FIG. 10A) by two-tailed student t-test, P value=0.0013 and (FIG. 10C) Mann Whitney test, P value=0.0059.

FIGS. 11A-11D depicts the identification of the immunogenic domains of Salp14. FIG. 11A shows that small peptides (approximately 20-25 amino acids) were synthesized that covered the entire Salp14 protein sequence (without signal peptide). ELISAs were performed to identify the fragment recognized by sera obtained from animals immunized with: FIG. 11B, salp14 mRNA-LNPs, FIG. 11C, salp14 DNA, FIG. 11D, Salp14 protein. The sequences of the fragments as well as the full-length Salp14 protein shown in FIG. 11A are also listed below:

Ixodes scapularis Salp14 protein sequence:
MGLTGTMLVLVSLAFFGSAAAHNCQNGTRPASEQDREGCDYYCWNAETK
SWDQFFFGNGEKCFYNSGDHGTCQNGECHLTNNSGGPNETDDYTPAPTE
KPKQKKKKTKKTKKPKRKSKKDQEKNL (SEQ ID NO: 1)
Ixodes scapularis Salp14 fragment 1:
HNCQNGTRPASEQDREGCDYY (SEQ ID NO: 2)
Ixodes scapularis Salp14 fragment 2:
GCDYYCWNAETKSWDQFFFG (SEQ ID NO: 3)
Ixodes scapularis Salp14 fragment 3:
QFFFGNGEKCFYNSGDHGTC (SEQ ID NO: 3)
Ixodes scapularis Salp14 fragment 4:
DHGTCQNGECHLTNNSGGPNETDD (SEQ ID NO: 4)
Ixodes scapularis Salp14 fragment 5:
PNETDDYTPAPTEKPKQKKK (SEQ ID NO: 5)
Ixodes scapularis Salp14 fragment 6:
KQKKKKTKKTKKPKRKSKKDQEKNL (SEQ ID NO: 6)

FIG. 12 depicts the results of the skin testing with Salp14 peptides. Skin tests identify the immunoreactive domain of Salp14 in tick immune animals. The synthetic peptides were injected intradermally into the skin of tick immune animals and monitored for erythema. The figure represents the skin reaction caused by Salp14, fragment 6 and bovine serum albumin (BSA) 48 hours after the injection.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

As disclosed herein, the ability of lipid nanoparticle-containing nucleoside-modified mRNAs encoding 19 I. scapularis salivary proteins (19ISP) (mRNAs encoding: Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09) to enhance recognition of a tick bite and diminish I. scapularis engorgement on a host—and thereby prevent B. burgdorferi infection was evaluated. Guinea pigs were immunized with 19ISP and challenged with I. scapularis. Animals administered 19ISP developed erythema at the bite site shortly after ticks began to attach, and these ticks fed poorly, marked by early detachment and decreased engorgement weights. 19ISP immunization also impeded B. burgdorferi transmission. The effective induction of local redness early after I. scapularis attachment and the inability of the ticks to take a normal blood meal, indicates that 19ISP may be used—either alone or in conjunction with traditional pathogen-based vaccines—for the prevention of Lyme disease, and potentially other tick-borne infections.

As disclosed herein, among the above nineteen (19) mRNAs that encode I. scapularis salivary proteins, seven (7) mRNAs result in relatively high antibody titer (mRNAs encoding: Salp14, Salp26A, TSLPI, IsPDIA3, TIX5, P32, and SG27). Ticks attached to guinea pigs immunized with these seven mRNAs have shorter attachment time and poorer feeding than ticks attached to guinea pigs immunized with medium titer-mRNAs, low titer-mRNAs or controls.

As demonstrated herein using the DNA, mRNA and protein of Salp 14 as a model, when an mRNA of a tick saliva protein can elicit an immune response, the tick saliva protein per se can also elicit an immune response at a lower but comparable level. Specifically, Salp14 was used as a model antigen to examine tick immunity using mRNA lipid nanoparticles (LNPs), plasmid DNA or recombinant protein platforms. salp14 containing mRNA-LNPs vaccination elicited erythema at the tick bite site after tick challenge that occurred earlier, and that was more pronounced, compared with DNA or protein immunizations. The Salp14 protein immunizations also elicited significant level of immunity. Humoral and cellular responses associated with tick immunity were towards a 25 amino acid region of Salp14 at the carboxy terminus of the protein.

One of ordinary skill in the art would understand that proteins are generally more stable than the mRNAs that translate into the proteins. Furthermore, protein-based compositions for generating immunity generally do not need potentially unstable delivery vehicles that need to be stored under low temperature conditions, which is often the case for mRNA-based compositions. As such, even though mRNAs of the tick saliva proteins elicit stronger immune responses, there are demands for tick saliva protein-based compositions for generating immunity and methods using the proteins to generate immunity.

Therefore, in some embodiments, the instant specification is directed to a method of generating tick immunity in a subject, the method including administering to the subject in need thereof a therapeutically effective amount of a tick-salivary protein. In some embodiments, the tick-salivary protein includes Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and/or SG09, or combinations thereof.

In some embodiments, the instant specification is directed to a composition for generating tick immunity in a subject, the composition including a therapeutically effective amount of a tick-salivary protein. In some embodiments, the tick-salivary protein includes at least one protein selected from the group consisting of: Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09.

Furthermore, although the instant specification describes the method of generating tick immunity and the composition for generating tick immunity using mainly the tick species Ixodes scapularis as an example, one of ordinary skill in the art would understand that the methods and compositions described herein are applicable to other species of ticks, as well. It is known to one of ordinary skill in the art that the resistances acquired from the bites of ticks of one species can elicit immunity against other tick species (see e.g., Lynn, G. et al., Am J Trop Med Hyg. 2021 January; 104(1):175-183. doi:10.4269/ajtmh.20-0776 (2021); which is incorporate herein in its entirety by reference). Therefore, the description of the instant specification is not limited to I. scapularis, but is rather applicable to various tick species.

### Definitions

Unless defined otherwise, all technical and scientific terms used herein have the same meaning as commonly understood by one of ordinary skill in the art to which the instant specification pertains. Although any methods and materials similar or equivalent to those described herein can be used in the practice for testing of the instant specification, selected materials and methods are described herein. In describing and claiming the instant specification, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably ±1%, and still more preferably ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the term “composition” or “pharmaceutical composition” refers to a mixture of at least one compound useful within the specification with a pharmaceutically acceptable carrier. The pharmaceutical composition facilitates administration of the compound to a patient or subject. Multiple techniques of administering a compound exist in the art including, but not limited to, intravenous, subcutaneous, oral, aerosol, parenteral, ophthalmic, pulmonary and topical administration.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the specification within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the specification, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the specification, and are physiologically acceptable to the patient. Supplementary active compounds may also be incorporated into the compositions. The “pharmaceutically acceptable carrier” may further include a pharmaceutically acceptable salt of the compound useful within the instant specification. Other additional ingredients that may be included in the pharmaceutical compositions used in the practice of the instant specification are known in the art and described, for example in Remington's Pharmaceutical Sciences (Genaro, Ed., Mack Publishing Co., 1985, Easton, PA), which is incorporated herein by reference.

As used herein, “tick-immunity” or “tick-resistance” are used interchangeably and refer to an immune response against one or more antigens involved in tick feeding. In certain embodiments, this response may include or be characterized by shorter tick feeding times and/or lower engorgement weight. In certain embodiments, hosts possessing tick-resistance or tick immunity may be less susceptible to or immune from tick-bite transmitted pathogens and conditions, including but not limited to Lyme disease, Anaplasma phagocytophilum, Powassan virus, A. phagocytophilum and Babesia microti.

As used herein, the terms “tick-salivary protein” or “SALP” may refer to any protein present in tick saliva.

As used herein, “treating a disease or disorder” means reducing the frequency with which a symptom of the disease or disorder is experienced by a patient. Disease and disorder are used interchangeably herein.

As used herein, the term “treatment” or “treating” encompasses therapy. Accordingly, the compositions and methods of the instant specification include therapeutic applications. Therefore “treating” or “treatment” of a state, disorder or condition includes: (i) inhibiting the state, disorder or condition, i.e., arresting or reducing the development of the disease or at least one clinical or subclinical symptom thereof, and/or (iii) relieving the disease, i.e. causing regression of the state, disorder or condition or at least one of its clinical or subclinical symptoms.

As used herein, the term “prevention” or “preventing” encompasses prophylaxis y. Accordingly, the compositions and methods of the instant specification include prophylactic applications. Therefore prevention” of or “preventing” a state, disorder or condition includes preventing or delaying the appearance of clinical symptoms of the state, disorder or condition developing in a subject that may be afflicted with or predisposed to the state, disorder or condition but does not yet experience or display clinical or subclinical symptoms of the state, disorder or condition.

Ranges: throughout this disclosure, various aspects can be presented in a range format. It should be understood that the description in range format is merely for convenience and brevity and should not be construed as an inflexible limitation on the scope of the instant specification. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Method of Generating Tick Immunity

Tick saliva is a complex blend of several proteins that are expressed dynamically depending on tick feeding and resulting changes in host responses. Using this information and previous analysis of the tick sialome, 19 salivary proteins were selected to form a cocktail of antigens (Table 1).

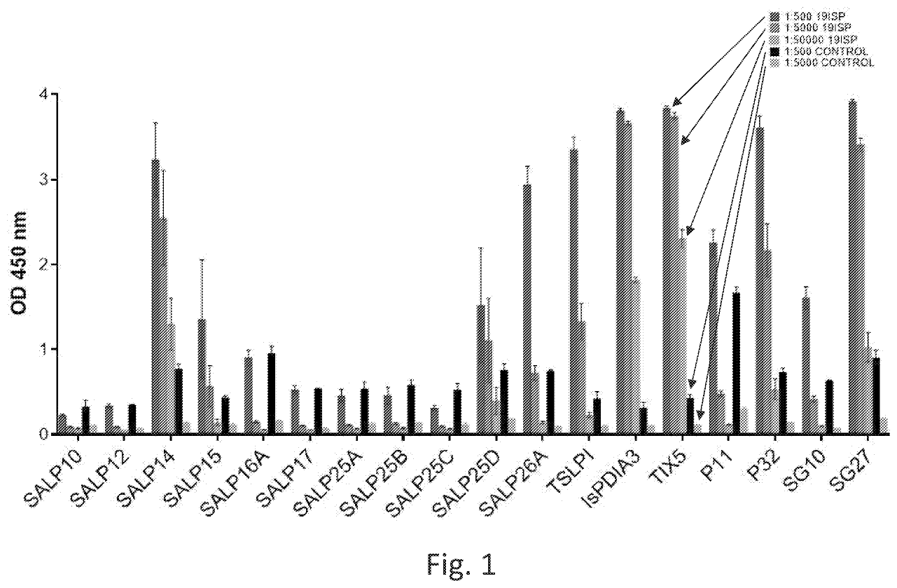

As demonstrated herein, immunizing a subject with a lipid nanoparticle-containing nucleoside-modified mRNAs encoding 19 I. scapularis salivary proteins (19ISP) (the 19 proteins are Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09) conferred the subject with robust immunity against tick bites. Further, robust antibody responses to a subset of the 19 antigens—Salp14, Salp15, Salp25D, Salp26A, TSLPI, IsPDIA3, TIX5, P32, SG10, and SG27—were detected in the 19ISP-immunized subjects. (See e.g., FIGS. 1-5F. Also discussed elsewhere herein.)

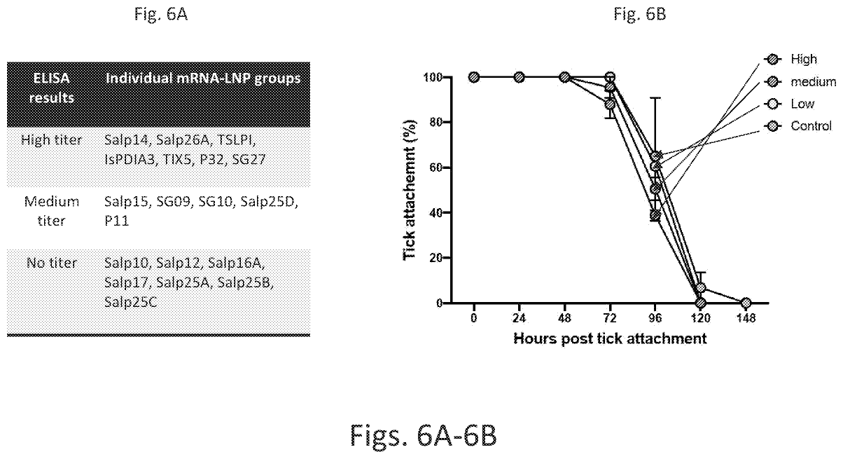

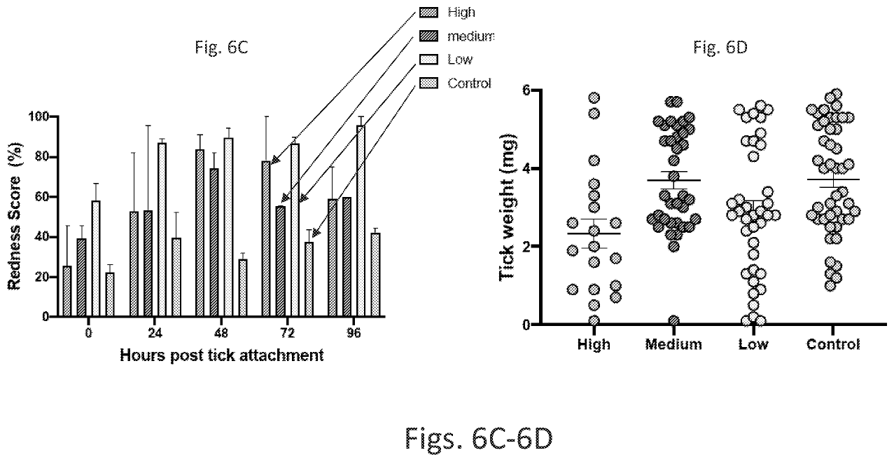

As demonstrated herein, immunizing subjects with a group of seven mRNAs (mRNAs of Salp14, Salp26A, TSLPI, IsPDIA3, TIX5, P32, and SG27), each of which resulted in high antibody titers in subjects immunized with 19ISP, resulted in significant tick immunity. (See e.g., FIGS. 6A-6D).

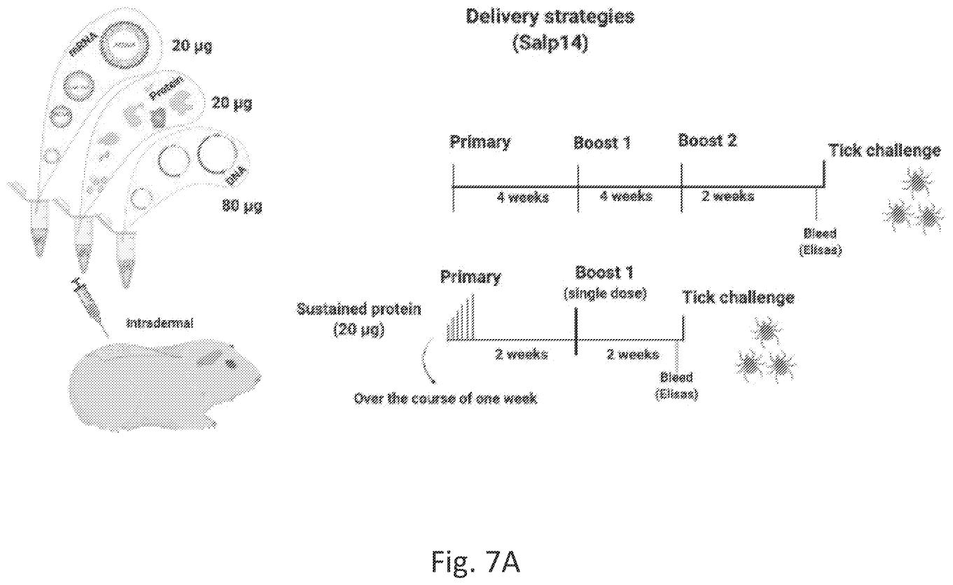

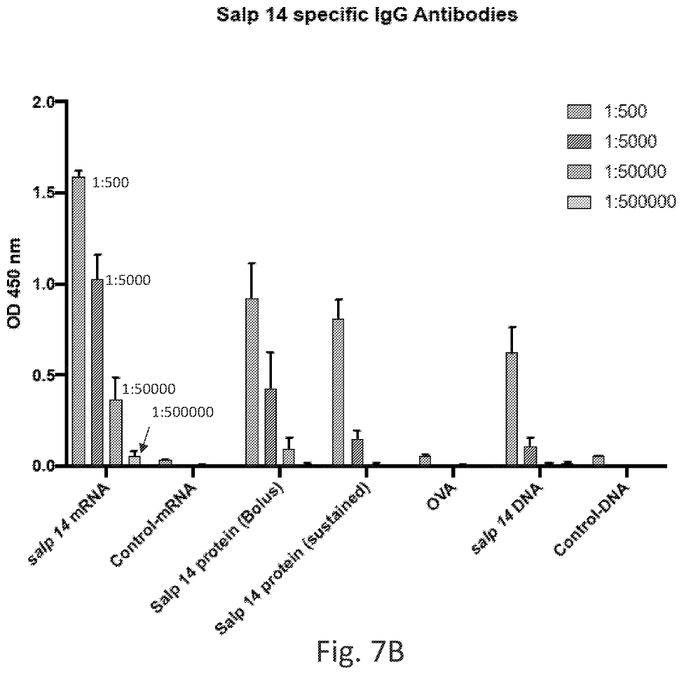

As demonstrated herein, immunizing subjects with an exemplary tick saliva protein (Salp14), as well as fragments thereof, elicited humoral responses comparable (although at a lower level than) to those caused by immunizing subjects with mRNAs of the same tick saliva protein and fragments. In certain embodiments, tick saliva proteins and the mRNAs of the same proteins elicit comparable level of immune responses. (See FIGS. 7A-12. Also discussed elsewhere herein.)

Therefore, in some embodiments, the instant specification is directed to a method of generating tick immunity in a subject. The method includes administering to the subject in need thereof a therapeutically effective amount of a tick-salivary protein, wherein the tick-salivary protein includes Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and/or SG09, or combinations thereof.

In some embodiments, the tick-salivary protein includes Salp10. In some embodiments, the tick-salivary protein includes Salp15. In some embodiments, the tick-salivary protein includes Salp25A. In some embodiments, the tick-salivary protein includes Salp25B. In some embodiments, the tick-salivary protein includes Salp25C. In some embodiments, the tick-salivary protein includes Salp25D. In some embodiments, the tick-salivary protein includes Salp14. In some embodiments, the tick-salivary protein includes TSLPI. In some embodiments, the tick-salivary protein includes Salp26A. In some embodiments, the tick-salivary protein includes P11. In some embodiments, the tick-salivary protein includes Salp16A. In some embodiments, the tick-salivary protein includes Salp17. In some embodiments, the tick-salivary protein includes TIX5. In some embodiments, the tick-salivary protein includes P32. In some embodiments, the tick-salivary protein includes Salp12. In some embodiments, the tick-salivary protein includes SG27. In some embodiments, the tick-salivary protein includes IsPDIA3. In some embodiments, the tick-salivary protein includes SG10. In some embodiments, the tick-salivary protein includes SG09.

In some embodiments, the tick-salivary protein includes IsPDIA3, TIX5, SG27, and Salp14. In some embodiments, the tick-salivary protein further includes Salp10, Salp15, Salp25A, Salp25B, Salp25C, Salp25D, TSLPI, Salp26A, P11, Salp16A, Salp17, P32, Salp12, SG10, and/or SG09, or combinations thereof.

In some embodiments, the tick-salivary protein includes IsPDIA3, TIX5, SG27, Salp14, and P32. In some embodiments, the tick-salivary protein further includes Salp10, Salp15, Salp25A, Salp25B, Salp25C, Salp25D, TSLPI, Salp26A, P11, Salp16A, Salp17, Salp12, SG10, and/or SG09, or combinations thereof.

In some embodiments, the tick-salivary protein includes IsPDIA3, TIX5, SG27, Salp14, P32, and TSLPI. In some embodiments, the tick-salivary protein further includes Salp10, Salp15, Salp25A, Salp25B, Salp25C, Salp25D, Salp26A, P11, Salp16A, Salp17, Salp12, SG10, and/or SG09, or combinations thereof.

In some embodiments, the tick-salivary protein includes Salp14, Salp26A, TSLPI, IsPDIA3, TIX5, P32, and SG27. In some embodiments, the tick tick-salivary protein further includes Salp10, Salp15, Salp25A, Salp25B, Salp25C, Salp25D, P11, Salp16A, Salp17, Salp12, SG10, and/or SG09, or combinations thereof.

In some embodiments, the tick-salivary protein includes Salp14, Salp15, Salp25D, Salp26A, TSLPI, IsPDIA3, TIX5, P32, SG10, and SG27. In some embodiments, the tick-salivary protein further includes Salp10, Salp25A, Salp25B, Salp25C, P11, Salp16A, Salp17, Salp12, and/or SG09, or combinations thereof.

In some embodiments, the tick-salivary protein includes Salp14, Salp15, Salp25D, Salp26A, TSLPI, IsPDIA3, TIX5, P32, SG10, SG27, SG09, and P11. In some embodiments, the tick-salivary protein further includes Salp10, Salp25A, Salp25B, Salp25C, Salp16A, Salp17, and/or Salp12, or combinations thereof.

In some embodiments, the tick-salivary protein includes Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09.

In some embodiments, the method further includes administering an adjuvant to the subject. In some embodiments, the adjuvant includes incomplete Freund's adjuvant, alum, addavax (equivalent to MF59), MF59, and/or AS03, or combinations thereof.

In some embodiments, the tick-salivary protein is administered by inhalational, oral, rectal, vaginal, parenteral, intracranial, topical, transdermal, intradermal, subcutaneous, pulmonary, intranasal, buccal, ophthalmic, intrathecal, intravenous, or combinations thereof.

In some embodiments, the subject is a mammal. In some embodiments, the subject is a human.

### Composition for Generating Tick Immunity

Tick saliva is a complex blend of several proteins that are expressed dynamically depending on tick feeding and resulting changes in host responses. Using this information and previous analysis of the tick sialome, the instant inventors selected 19 salivary proteins to form a cocktail of antigens (Table 1).

As demonstrated herein, immunizing a subject with a lipid nanoparticle-containing nucleoside-modified mRNAs encoding 19 I. scapularis salivary proteins (19ISP) (the 19 proteins are Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09) conferred the subject with robust immunity against tick bites. As demonstrated herein, robust antibody responses to a subset of the 19 antigens—Salp14, Salp15, Salp25D, Salp26A, TSLPI, IsPDIA3, TIX5, P32, SG10, and SG27—were detected in the 19ISP-immunized subjects. (See e.g., FIGS. 1-5F. Also discussed elsewhere herein.)

As demonstrated herein, immunizing subjects with a group of seven mRNAs (mRNAs of Salp14, Salp26A, TSLPI, IsPDIA3, TIX5, P32, and SG27), each of which resulted in high antibody titers in subjects immunized with 19ISP, resulted in significant tick immunity. (See e.g., FIGS. 6A-6D).

As demonstrated herein, immunizing subjects with an exemplary tick saliva protein (Salp14), as well as fragments thereof, elicited humoral responses comparable (although at a lower level than) to those caused by immunizing subjects with mRNAs of the same tick saliva protein and fragments, indicating that tick saliva proteins and the mRNAs of the same proteins elicit comparable level of immune responses. (See FIGS. 7A-12. Also discussed elsewhere herein.)

Therefore, in some embodiments, the instant specification is directed to a composition including a therapeutically effective amount of a tick-salivary protein, wherein the tick-salivary protein includes Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and/or SG09, or combinations thereof.

In some embodiments, the composition is a pharmaceutical composition for generating tick immunity in a subject. In some embodiments, the subject is a mammal. In some embodiments, the subject is a human.

In some embodiments, the tick-salivary protein includes IsPDIA3, TIX5, SG27, and Salp14. In some embodiments, the tick-salivary protein further includes Salp10, Salp15, Salp25A, Salp25B, Salp25C, Salp25D, TSLPI, Salp26A, P11, Salp16A, Salp17, P32, Salp12, SG10, and/or SG09, or combinations thereof.

In some embodiments, the tick-salivary protein includes IsPDIA3, TIX5, SG27, Salp14 and P32. In some embodiments, the tick-salivary protein further includes Salp10, Salp15, Salp25A, Salp25B, Salp25C, Salp25D, TSLPI, Salp26A, P11, Salp16A, Salp17, Salp12, SG10, SG09, or combinations thereof.

In some embodiments, the tick-salivary protein includes IsPDIA3, TIX5, SG27, Salp14, P32, and TSLPI. In some embodiments, the tick-salivary protein further includes Salp10, Salp15, Salp25A, Salp25B, Salp25C, Salp25D, Salp26A, P11, Salp16A, Salp17, Salp12, SG10, and/or SG09, or combinations thereof.

In some embodiments, the tick-salivary protein includes Salp14, Salp26A, TSLPI, IsPDIA3, TIX5, P32, and SG27. In some embodiments, the tick tick-salivary protein further includes Salp10, Salp15, Salp25A, Salp25B, Salp25C, Salp25D, P11, Salp16A, Salp17, Salp12, SG10, and/or SG09, or combinations thereof.

In some embodiments, the tick-salivary protein includes Salp14, Salp15, Salp25D, Salp26A, TSLPI, IsPDIA3, TIX5, P32, SG10, and SG27. In some embodiments, the tick-salivary protein further includes Salp10, Salp25A, Salp25B, Salp25C, P11, Salp16A, Salp17, Salp12, and/or SG09, or combinations thereof.

In some embodiments, the tick-salivary protein includes Salp14, Salp15, Salp25D, Salp26A, TSLPI, IsPDIA3, TIX5, P32, SG10, SG27, SG09, and P11. In some embodiments, the tick-salivary protein further includes Salp10, Salp25A, Salp25B, Salp25C, Salp16A, Salp17, and/or Salp12, or combinations thereof.

In some embodiments, the tick-salivary protein includes Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09.

In some embodiments, the composition further includes an adjuvant. In some embodiments, the adjuvant includes incomplete Freund's adjuvant, alum, addavax (equivalent to MF59), MF59, AS03, or combinations thereof.

In some embodiments, the composition is administered by inhalational, oral, rectal, vaginal, parenteral, intracranial, topical, transdermal, intradermal, subcutaneous, pulmonary, intranasal, buccal, ophthalmic, intrathecal, intravenous, or combinations thereof.

### Administration/Dosing

In clinical settings, delivery systems for the compositions described herein can be introduced into a subject by any of a number of methods, each of which is familiar in the art. For instance, a pharmaceutical formulation of the composition can be administered by inhalation or systemically, e.g. by intravenous injection.

The regimen of administration may affect what constitutes an effective amount. The therapeutic formulations may be administered to the subject either prior to or after the manifestation of symptoms associated with the disease or condition. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the composition of the instant specification to a subject, preferably a mammal, more preferably a human, may be carried out using known procedures, at dosages and for periods of time effective to treat a disease or condition in the subject. An effective amount of the composition necessary to achieve a therapeutic effect may vary according to factors such as the time of administration; the duration of administration; other drugs, compounds or materials used in combination with the composition; the state of the disease or disorder; age, sex, weight, condition, general health and prior medical history of the subject being treated; and like factors well-known in the medical arts. Dosage regimens may be adjusted to provide the optimum therapeutic response. For example, several divided doses may be administered daily or the dose may be proportionally reduced as indicated by the exigencies of the therapeutic situation. One of ordinary skill in the art would be able to study the relevant factors and make the determination regarding the effective amount of the composition without undue experimentation. Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the instant specification include oral, nasal, rectal, intravaginal, parenteral, buccal, sublingual or topical. The compounds for use in the instant specification may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the instant specification are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the instant specification may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds of the instant specification may be formulated for injection or infusion, for example, intravenous, intramuscular, or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Controlled Release Formulations and Drug Delivery Systems

In certain embodiments, the formulations of the instant specification may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material that provides sustained release properties to the compounds. As such, the compounds for use the method of the instant specification may be administered in the form of microparticles, for example, by injection or in the form of wafers or discs by implantation.

In certain embodiments, the compounds of the instant specification are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the instant specification depends on the age, sex and weight of the patient, the current medical condition of the patient and the progression of a disease or disorder contemplated herein in the patient being treated. The skilled artisan is able to determine appropriate dosages depending on these and other factors.

A suitable dose of a compound of the instant specification may be in the range of from about 0.001 mg to about 5,000 mg per day, such as from about 0.01 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

Actual dosage levels of the cells in the pharmaceutical formulations of the instant specification may be varied so as to obtain an amount of the composition that are effective to achieve the desired therapeutic response for a particular subject, composition, and mode of administration, without being toxic to the subject.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLES

The instant specification further describes in detail by reference to the following experimental examples. These examples are provided for purposes of illustration only, and are not intended to be limiting unless otherwise specified. Thus, the instant specification should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the instant specification and practice the claimed methods. The following working examples therefore, specifically point out selected embodiments of the instant specification, and are not to be construed as limiting in any way the remainder of the disclosure.

### Example 1: Generation of the Lipid Nanoparticle-Containing Nucleoside-Modified mRNAs Encoding 19 I. scapularis Salivary Proteins (19ISP)

19 genes known to be expressed in I. scapularis salivary glands, many of which are secreted at the bite site, were selected for this study (Table 1). Salivary protein of 14 kDa (Salp14), tick lectin pathway inhibitor (TSLPI), Salp10, Salp15, Salp16A, Salp17, Salp25A, Salp25B, Salp25C, Salp25D, Salp26A, tick inhibitor of factor Xa (TIX5), and a 32 kDa salivary protein (P32) were initially identified by immunoscreening assays as secreted salivary proteins that reacted avidly with tick-resistant animal sera. Some of these salivary antigens regulate host immune responses, or influence pathogen infectivity. P11 is a secreted salivary protein involved in A. phagocytophilum infection of salivary glands. Salp12, SG09, SG10, SG27, and I. scapularis protein disulfide isomerase (IsPDIA3) are secreted salivary proteins that may influence B. burgdorferi acquisition. SG10 is a heme lipoprotein and SG09 is a hemelipoglyco-carrier protein, present in I. scapularis saliva with homologs identified in saliva, hemolymph, and tissues of a variety of other tick species, including Ixodes ricinus. Heme-binding class proteins are highly abundant in the saliva of I. scapularis, with known or putative functions in other tick species that include transport and storage of heme, detoxification, and involvement in innate immunity. A recent study included an I. ricinus heme lipoprotein homologous to SG10 among the many potential targets that may influence tick infestation in rabbits and dogs.

TABLE 1
Genes incorporated into the 19ISP mRNA vaccine

| | Gene/protein | |
|---|---|---|
| Gene | accession | Characteristics^{a} |
| Salp10 | AAK97828/ | Elicits antibodies in host, homology with Kunitz-type |
| | AF278575 | protease inhibitors, altered expression in response to |
| | | A. phagocytophilum |
| Salp15 | AAK97817/ | Alters CD4⁺ T cell function, influences B. burgdorferi |
| | AF209914 | infection |
| Salp25A | AAK97825/ | Generates antibodies in host, putative histamine binding |
| | AF209922 | protein |
| Salp25B | AAK97821/ | Produces antibodies in host, probable histamine binding |
| | AF209918 | protein |
| Salp25C | AAK97816/ | Potential histamine binding protein, altered expression in |
| | AF209913 | response to A. phagocytophilum |
| Salp25D | AAK97814/ | Peroxiredoxin, role in acquisition of B. burgdorferi by |
| | AF209911 | I. scapularis in the host |
| Salp14 | AAK97824/ | Anticoagulant that inhibits factor Xa |
| | AF209921 | |
| TSLPI | HQ605983/ | Lectin complement pathway inhibition |
| | AEE89466 | |
| Salp26A | AAK97822/ | Generates antibodies in host |
| | AF209919 | |
| P11 | DQ066011/ | Facilitates A. phagocytophilum migration to tick salivary |
| | AAY66648 | glands |
| Salp16A | AAK97818/ | Elicits antibodies in host, homology with Salp15 |
| | AF209915 | |
| Salp17 | AAK97819/ | Expression decreased by A. phagocytophilum |
| | AF209916 | |
| TIX5 | AEE89467/ | Factor Xa Inhibitor |
| | HQ605984 | |
| P32 | ADO95260/ | Reacts with tick immune serum |
| | HM802761 | |
| Salp12 | XP_002433874/ | Chemoattractant for B. burgdorferi and influences |
| | XM_002433829 | acquisition |
| SG27 | XP_002405832/ | Putative B. burgdorferi attractant, protein disulfide- |
| | XM_002405788 | isomerase |
| IsPDIA3 | XP_002406442/ | Protein disulfide-isomerase, Contributes to |
| | XM_002406398 | B. burgdorferi colonization of ticks |
| SG10 | XP_002411436/ | Heme lipoprotein |
| | XM_002411391 | |
| SG09 | XP_002411435/ | Hemelipoglyco-carrier protein |
| | XM_002411390 | |

^{a}Data from references, Pfam and Simple modular architecture research tool (SMART) domain analysis

The individual nucleoside-modified mRNAs for 19 genes were synthesized in vitro and encapsulated in a lipid nanoparticle (LNP) in equal amounts to generate 19ISP, as outlined in the methods section. The 19ISP mRNA-LNP vaccine was used to immunize guinea pigs to test for the generation of immunity against tick bites.

### Example 2: Immunization with 19ISP Generates Antibody Responses to Specific Antigens in Guinea Pigs

Guinea pigs were immunized intradermally three times at 4-week intervals with 50 μg 19ISP mRNA-LNP and IL-21 mRNA-LNP as a control. Two weeks after the last dose and prior to tick challenge, blood was collected from the immunized guinea pigs and sera were isolated. Sera IgG titers were evaluated by ELISA using recombinant salivary protein antigens. Eighteen antigens were tested for the presence of specific antibodies. The primary sequences of SG09 and SG10 share 75% identity which precludes conclusive determination of antibodies specific to these two proteins. Therefore, recombinant SG09 was not generated for ELISA assays. Interestingly, antibodies were detected against 10 of the selected proteins—Salp14, Salp15, Salp25D, Salp26A, TSLPI, IsPDIA3, TIX5, P32, SG10, and SG27 (FIG. 1). Antibodies for these proteins were not detected with the sera obtained from the control animals. These results suggest that 19ISP vaccination elicits antigen-specific humoral responses in guinea pigs.

### Example 3: 19ISP Immunization Elicits Protective Responses Against Tick Challenge in Guinea Pigs

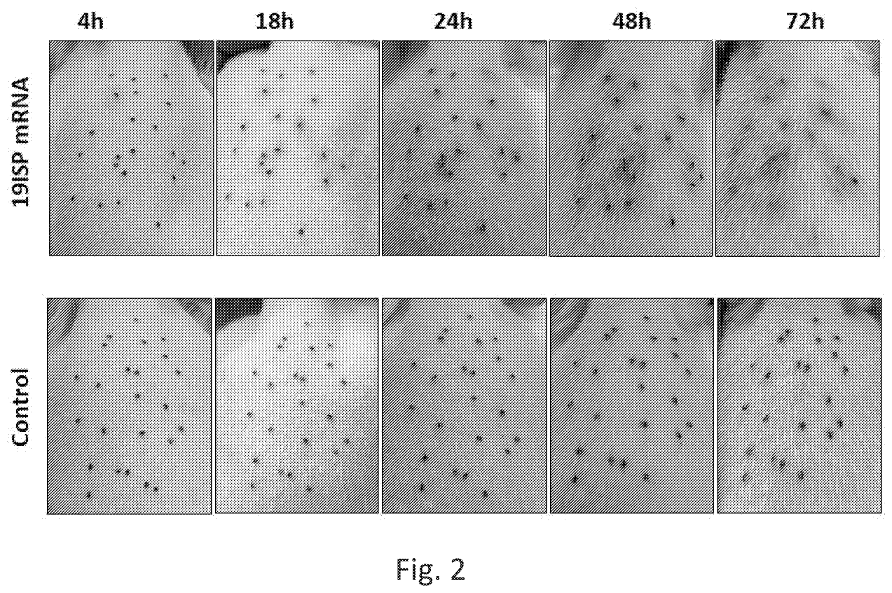

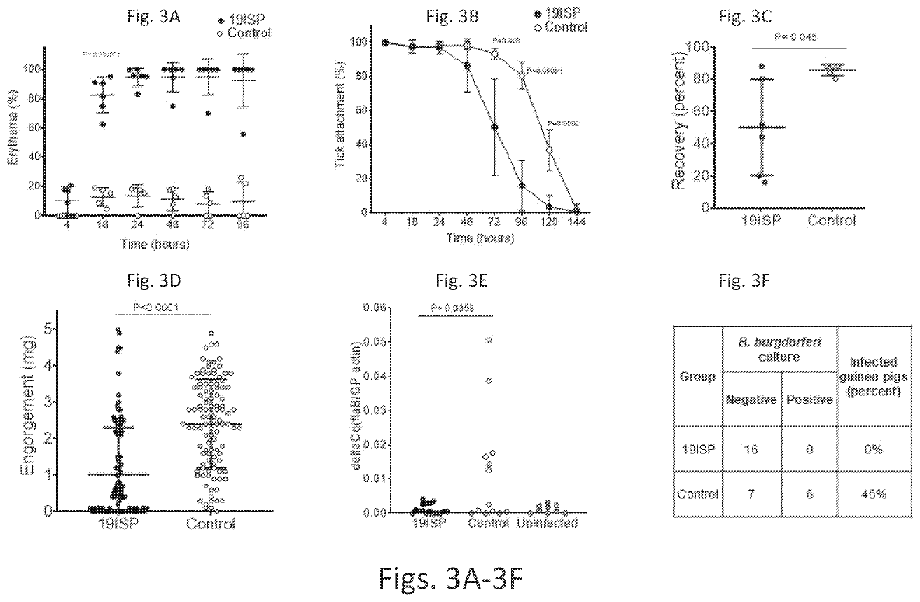

25 uninfected I. scapularis nymphs were placed on 19ISP-immunized or control (IL21-immunized) guinea pigs and allowed to naturally attach. The guinea pigs were monitored for the development of erythema at the bite site, which is the earliest hallmark associated with acquired tick resistance. As shown in FIG. 2, significant erythema was observed in 19ISP-immunized animals as early as 18 hours post tick challenge. The erythema peaked at 24 hours at all the bite sites and persisted throughout the tick challenge. Tick bite in the control animals did not show substantial redness (FIGS. 2 and 3A).

The immunized guinea pigs were also monitored for other hallmarks of tick immunity that occur after the appearance of erythema, including tick rejection, feeding, and engorgement weights. For 19ISP-immunized animals, the ticks fed poorly and started to detach by 48 hours post tick challenge (FIG. 3B). Being small in size and poorly fed, the recovery of I. scapularis was also reduced with many of the dead tick shells merely attached to the guinea pigs (see FIG. 2, 72h panel, and FIG. 3C). By 96 hours post tick challenge, 80% of the ticks detached from the 19ISP-immunized guinea pigs, as compared to 20% detachment in control animals. Due to poor feeding and early detachment, the engorgement weights of the ticks fed upon 19ISP-immunized animals were significantly lower (mean weight=1.02 mg, n=109) as compared to the ticks which fed upon control animals (mean weight=2.42 mg, n=108) (FIG. 3D). These data indicate that 19ISP vaccination induces acquired resistance to tick bites in guinea pigs.

A subsequent study was performed to determine whether 19ISP immunization could influence the transmission of B. burgdorferi to guinea pigs. Although a single B. burgdorferi-infected tick is sufficient to transmit full infection, three B. burgdorferi-infected I. scapularis nymphs were placed on each guinea pig immunized either with 19ISP or Luc-mRNA control. As most humans do not commonly get 3 tick bites at the same time, and because many I. scapularis in nature are not infected with B. burgdorferi, this study assumed that 3 ticks on guinea pigs represent a substantial degree of exposure to the Lyme disease agent. In addition, as humans are likely to remove a tick that causes a bite with concomitant erythema or pruritis, the instant inventors removed the ticks from the experimental and control guinea pigs, in a double-blind manner, when redness became evident. At 3 weeks after exposure to ticks, the guinea pigs were euthanized, and biopsies were taken adjacent to bite site to test the infection levels. The experiment was repeated three times. In total, almost half (46%) of the control guinea pigs were PCR-positive (6/13) for B. burgdorferi while none (0%) of the 19ISP-immunized guinea pigs were PCR-positive (0/16) for B. burgdorferi (FIG. 3E). To further confirm these results, B. burgdorferi was cultured from skin biopsy specimens from all PCR-positive guinea pigs. All cultures were negative from PCR-negative animals (FIG. 3F). In a subsequent experiment a single B. burgdorferi-infected tick was allowed to feed without removal on 19ISP-immunized or control guinea pigs, to mimicking an un-noticed human infection from single tick bite. It was found that 60% (3/5) of control guinea pigs were infected with B. burgdorferi, whereas none of the 19ISP-immunized animals were infected, as tested by PCR and culture. Collectively, these data show that 19ISP-immunization can protect against tick-borne B. burgdorferi infection.

### Example 4: Differential Host Responses Associated with 19ISP-mRNA Vaccination

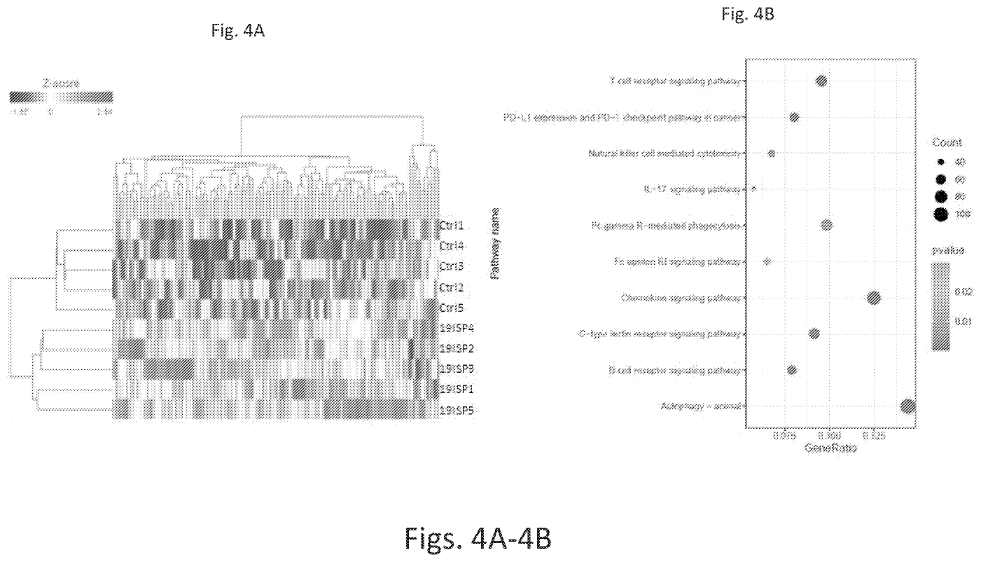

To further understand the immune responses associated with I9ISP vaccination and host protection, RNA from immunized guinea pigs was isolated for gene expression analysis two weeks after the final immunization. Significant differences in whole blood gene expression in 19ISP-immunized animals were found as compared to controls. Principal component analysis (PCA) and cluster dendrogram revealed that the 19ISP-vaccinated animal group formed a separate cluster from the control animal groups (data not shown). A total of 125 differentially expressed genes were identified with a p-value less than 0.05 and a fold change greater than or equal to 2.0. 113 genes were up-regulated, and 12 genes were down-regulated in the 19ISP-immunized group as compared to the control group (FIG. 4A). To identify signaling pathways involved in response to 19ISP-mRNA vaccination, KEGG pathway enrichment analysis was utilized. The top 20 enriched pathways include many immune pathways The top immune pathways enriched following vaccination are T cell receptor, B-cell receptor signaling pathways, chemokine signaling pathways, IL-17 signaling, Natural killer cell-mediated cytotoxicity, FcεRI-mediated signaling, and C type lectin receptor signaling pathways (FIG. 4B). In addition, gene expression at the erythematic bite site was also compared with a non-erythematic site in the same guinea pigs immunized with 19ISP. The data showed enrichment of T-cell-related pathways, indicating elicited T-cell response.

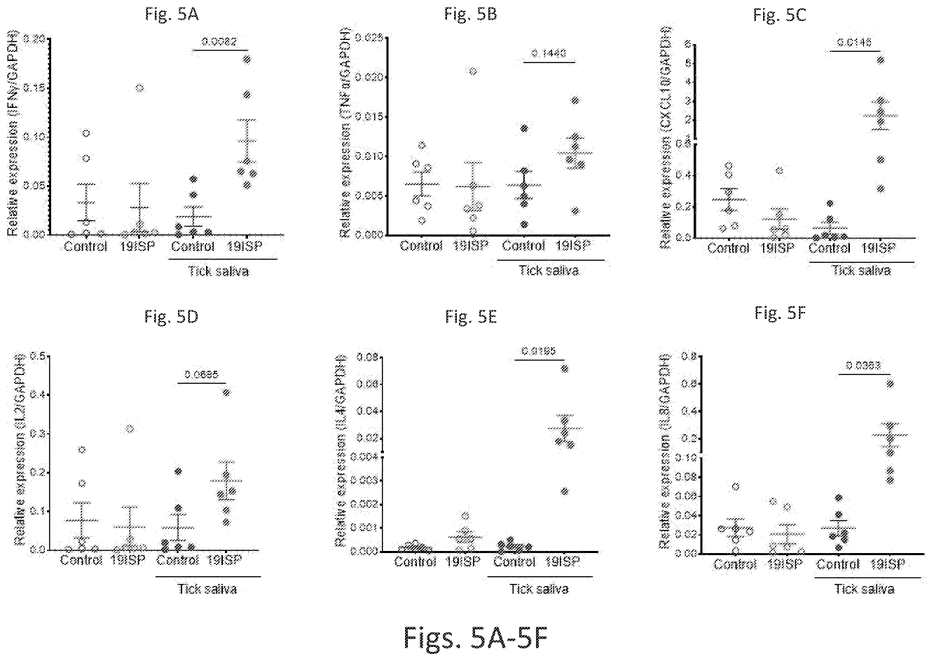

To further understand some of the cell-mediated immune responses due to the 19ISP vaccination, PBMCs were isolated from 19ISP- and control (Luc)-mRNA immunized guinea pigs, 2-weeks after the second boost. PBMCs were stimulated with I. scapularis saliva, total RNA was extracted, and the expression of selected cytokines/chemokines commonly induced by activated T- and B-cells following vaccinations, including IFNγ, TNFα, CXCL10, IL2, IL4, and IL8, were examined. The expression of these cytokines was increased in 19ISP-immunized animals (FIGS. 5A-5F) as compared to controls.

### Example 5: Selected Discussion

In the study detailed elsewhere herein, 19 salivary proteins that have a spectrum of functions in tick feeding, interaction with the pathogen, or host responses (reflecting a portion of the tick sialome; Table 1) were investigated towards generation of acquired tick resistance or “tick immunity” to I. scapularis. To optimize the immune response, a nucleoside-modified mRNA-LNP platform that allows for more continuous delivery of the antigen was chosen. In certain non-limiting embodiments, this allows for more close resemblance to a tick bite. Safe and effective nucleoside-modified mRNA-LNP vaccines are currently being used in humans against SARS-CoV-2, therefore mRNA-LNP vaccines are a good vaccine platform to deliver tick antigens to a host.

Guinea pigs were used as the primary animal model because they are not part of the natural life cycle of I. scapularis and readily develop tick immunity following repeated exposure to I. scapularis. In addition, guinea pigs can be infected with tick-borne B. burgdorferi, and can therefore be used to determine whether acquired tick resistance can influence the transmission of the Lyme disease agent. Immunization with 19ISP provided robust tick immunity in guinea pigs, including significant early erythema after tick placement on the animals and rapid tick detachment, along with severely impaired tick feeding and low engorgement weights.

Host responses in the 191SP-immunized guinea pigs were further assessed, using the relatively limited reagents for this species. Robust antibody responses to a subset of the 19 antigens—Salp14, Salp15, Salp25D, Salp26A, TSLPI, IsPDIA3, TIX5, P32, SG10, and SG27—were detected in 19ISP-immunized guinea pigs, suggesting that these may be the targets most closely associated with tick immunity (FIG. 1). Nine of the proteins did not elicit detectable responses. Without wishing to be limited by any theory, this suggests that they are not highly antigenic and may not be directly linked with tick immunity (FIG. 1). PBMCs from 19ISP-immunized guinea pigs stimulated with I. scapularis saliva elicited the production of several T-cell-related cytokines (FIGS. 5A-5F). Without wishing to be limited by any theory, this suggests that a combination of humoral and cell-mediated immunity accounts for the magnitude of response by 19ISP mRNA vaccination. Finally, RNA-seq elucidated the specific genetic signatures associated with 19ISP-immunization in guinea pigs. The activated pathways included T cell receptor, B-cell receptor signaling pathways, chemokine signaling pathways, IL-17 signaling, natural killer cell-mediated cytotoxicity, FcεRI mediated signaling, and C-type lectin receptor signaling pathways. FcεRI signaling and B-cell receptor signaling pathways are also activated at the bite site of guinea pigs with naturally acquired tick resistance following exposure to multiple tick-bites (C. Kurokawa et al., Ticks Tick Borne Dis 11, 101529 (2020)). The activation of common pathways in naturally acquired tick resistance and after 19ISP-immunization can help to guide vaccine development and studies with mRNA LNPs containing selected subsets of the genes help address the contribution of each of the 19 genes in the genesis of acquired tick resistance.

The I9ISP-immunized guinea pigs were protected from tick-borne B. burgdorferi infection when the ticks were removed when erythema became pronounced. This time point was chosen because when humans notice redness or irritation due to a tick bite, the immediate response is to remove the tick. Such erythema-associated itch is apparent in tick immune guinea pigs and likely to occur in humans. Additionally, when challenged with a B. burgdorferi-infected tick that was allowed to continue to try to feed until it fell off, none of 191SP immunized animals were infected, while 60% of the control guinea pigs were infected. Humans, like guinea pigs, are not important in the natural life cycle of I. scapularis, and therefore vaccination of humans with 191SP can help prevent Lyme disease, either because the ticks naturally detach early or because humans more readily remove ticks when erythema is present. Ticks that attach and feed to repletion on humans are much more likely to transmit B. burgdorferi than ticks that are removed early in the feeding process.

In conclusion, 191SP immunization can elicit acquired resistance against I. scapularis and prevent tick-borne B. burgdorferi infection in guinea pigs. In certain embodiments, protection extends to other I. scapularis-borne pathogens, such as Babesia microti, Anaplasma phagocytophilum, and Powassan virus, among others. These data demonstrate that a multivalent vaccine that targets I. scapularis antigens has the potential to prevent a common tick-borne infectious disease.

### Example 6: Materials and Methods for Examples 1-4

### Ticks and Animals

I. scapularis ticks were obtained from Oklahoma State University, Stillwater, OK, and maintained in an incubator at 23° C. and 90% relative humidity under a 14 h light, 10 h dark photoperiod. 4-5-weeks old female Hartley guinea pigs (Charles River Laboratories, MA) were used to feed nymphal ticks. Six weeks old female C3H mice (Charles River Laboratories, MA) were used for tick infection.

### Preparation of 191SP mRNA-Lipid Nanoparticle (mRNA-LNP)

mRNA-LNPs were generated as previously described (A. W. Freyn et al., Mol Ther 28, 1569-1584 (2020), the entirety of which is incorporated herein by reference). mRNA vaccines encoding individual salivary antigens with their own signal peptide or IL2-signal peptide, and IL21 or firefly luciferase (Luc) were codon-optimized, synthesized, and cloned into the mRNA production plasmid as described (Freyn et al., 2020). mRNA production and LNP encapsulation was performed as described (Freyn et al., 2020). Briefly, the sequence of mRNAs was transcribed to contain 101 nucleotide-long poly(A) tails. m1Ψ-5′-triphosphate (TriLink) instead of UTP was used to generate modified nucleoside-containing mRNA. Capping of the in vitro transcribed mRNAs was performed co-transcriptionally using the trinucleotide cap1 analog, CleanCap (TriLink). mRNAs were purified by cellulose purification, as previously described (M. Baiersdorfer et al., Mol Ther Nucleic Acids 15, 26-35 (2019), the entirety of which is incorporated herein by reference). All mRNAs were analyzed by agarose gel electrophoresis and were stored frozen at −20° C. In the multivalent 19ISP formulation equal amounts (by weight) from each of the 19 mRNAs were combined prior to LNP formulation. mRNAs were encapsulated in LNPs using a self-assembly process in which an aqueous solution of mRNA at acidic pH 4.0 was rapidly mixed with a solution of lipids dissolved in ethanol (M. A. Maier et al., Mol Ther 21, 1570-1578 (2013) and M. Jayaraman et al, Angew Chem Int Ed Engl 51, 8529-8533 (2012), the entireties of which are hereby incorporated herein by reference), which contain an ionizable cationic lipid/phosphatidylcholine/cholesterol/PEG-lipid(50:10:38.5:1.5 mol/mol) and were encapsulated at an RNA to total lipid ratio of ˜0.05 (wt/wt), were stored at −80° C. at a concentration of mRNA of ˜1 μg/μl . The LNPs had a diameter of ˜80 nm as measured by dynamic light scattering using a Zetasizer Nano ZS (Malvern Instruments Ltd, Malvern, UK) instrument, with a polydispersity index of 0.02-0.06 and an encapsulation efficiency of ˜95%.

Two or three batches from each mRNA-LNP formulations were used in these studies and variability was not observed in vaccine efficacy. LNPs used in this study is proprietary to Acuitas Therapeutics; the proprietary lipid and LNP composition are described in U.S. Pat. No. 10,221,127, the entirety of which is hereby incorporated herein by reference.

### Immunization of Guinea Pigs

Female Hartley guinea pigs (5-weeks old) were immunized intradermally with 50 μg of 19ISP mRNA-LNPs (˜2.63 μg per antigen) or murine IL-21/Firefly Luciferase (Luc) mRNA-LNP (control). Since, a combination of 19 antigens has never been used, the approximate dosage was estimated on the basis of previous studies performed in mice and guinea pigs. Intradermal immunization is more efficient when using lower doses of antigens, without compromising the efficacy. The required amount of frozen mRNA-LNPs were thawed at room temperature, diluted with sterile PBS and used within 2 hours for injection. The animals were boosted twice at 4-week intervals. The animals were bled retro-orbitally 2 weeks after the last immunization to obtain blood for RNAseq and the serum was separated for use in ELISA. A minimum of 3 animals were used in each group.

### Generation of Recombinant Salivary Proteins

RNA was isolated from salivary glands dissected from I. scapularis ticks fed to repletion and cDNA synthesized according to the manufacture's protocol (iScript cDNA synthesis kit, Bio-Rad). Gene-specific primers were used to amplify the mRNA region encoding the mature proteins listed in Table 1. Purified amplicons were then cloned into the pMT-Bip-V5-HisA cloning vector or the pET28A Escherichia coli expression vector and recombinant DNA sequenced at the Keck sequencing facility, Yale University, to validate the clones. Recombinant proteins were generated using a Drosophila expression system as described earlier for different antigens. For expression in E. coli, the clones were transformed in endotoxin-free ClearColi cells BL21(DE3) (Lucigen, WI) and expressed with an N-terminal His₆-tag. The proteins were purified using Ni²⁺-NTA-Agarose resin according to the manufacturer's instructions (Qiagen, CA). Protein purity was assessed by SDS-PAGE using 4-20% gradient precast gels (Biorad, CA) and quantified using the BCA protein estimation kit (Thermo Scientific, MA). Recombinant IsPDIA3 was generated as a GST-fusion (glutathione-S-transferase) protein in E. coli using the pGEX-4T2 vector and recombinant protein purified using GST-resin according to the manufacturer's protocol (GE Healthcare Life Sciences, PA).

### ELISA Assessment of Recombinant Salivary Proteins

To assess the 19ISP specific antibody response against individual proteins, 96-well Immunosorp ELISA plates were coated overnight with 250 ng of recombinant proteins, blocked with 3% BSA for 1 h at 37° C. and incubated with guinea pig anti-19ISP sera collected 2 weeks after the last immunization dose at 1:500, 1:5,000 or 1:50,000 dilutions for 2 h. Each step was separated by 3 washes with PBST (PBS with 0.025% Tween-20) Bound antibody was detected with HRP-conjugated goat anti-guinea pig IgG secondary antibody and TMB substrate solution (ThermoFisher Scientific, IL). The reaction was stopped by TMB stop solution and absorbance was read at 450 nm.

### RNAseq Analysis

Total RNA was extracted from whole blood obtained from guinea pigs 2 weeks after the final immunization. Trizol was added to the whole blood and RNA was isolated according to the manufacturer's instructions (Qiagen, CA). RNA was submitted for library preparation using TruSeq (Illumina, San Diego, CA, USA) and sequenced using Illumina HiSeq 2500 by paired-end sequencing at the Yale Center for Genome Analysis (YCGA). RNAseq analyses including alignment, quantitation, normalization, and differential gene expression analyses were performed using Partek Genomics Flow software (St. Louis, MO, USA). Specifically, RNAseq data were trimmed and aligned to the guinea pig genome (Cavea porcellus, Cavpor 3.0 from Ensembl), with associated annotation file using STAR (v2.7.3a) (Dobin et al., 2013). The aligned reads were quantified to Ensembl transcripts using the Partek E/M algorithm (Xing et al., 2006) and the subsequent steps were performed on gene-level annotation followed by total count normalization. The gene-level data were normalized by dividing the gene counts by the total number of reads followed by the addition of a small offset (0.0001). Principal components analysis (PCA) was performed using default parameters for the determination of the component number, with all components contributing equally in Partek Flow. Hierarchal clustering was performed on the genes that were differentially expressed across the conditions (P<0.05, fold change ≥2 for each comparison). Pathway enrichment was conducted by converting the guinea pigs Ensembl gene symbol to the Entrez gene ID for mice as described in Kurokawa et al. (2020), since the guinea pig genome is not annotated in Partek Flow. The top 10 immune pathways were further plotted on a bubble diagram by ggplot2 in R studio.

Cytokine Expression Analysis from Peripheral Blood Mononuclear Cells (PBMCs)

PBMCs were isolated from guinea pig blood 2 weeks after third immunization and stimulated with 3 μl of tick saliva in a total volume of 100 μl for 24 h at 37° C. RNA was extracted from stimulated and unstimulated PBMCs using the Qiagen RNeasy kit and cDNA was prepared from the purified RNA using the iScript cDNA synthesis kit (BioRad). cDNA was analyzed by quantitative RT-PCR using the iTaq Sybr Green Supermix (Biorad, CA) for the expression of guinea pig-specific cytokines and chemokines, including interferon-γ (IFNγ), tumor necrosis factor-α (TNFα), CXCL10, Interleukin 2 (IL-2), IL-4 and IL-8. The relative expression was calculated using the deltaCq method and normalized to the expression of the guinea pig gapdh gene.

### Uninfected Tick Challenge of Guinea Pigs

Two weeks following the last immunization dose, guinea pigs were challenged with uninfected I. scapularis ticks. Briefly, after anesthetizing by intramuscular injection of a ketamine and xylazine mixture, the guinea pigs were challenged with 25 I. scapularis nymphs. Ticks were allowed to attach to shaved backs of guinea pigs. Guinea pigs were housed individually with 3 layers of tick containment (a pan of water below the wire-bottom of the cage, a hopper-inclusive lid, and grease around the outer edges of the cage). Daily monitoring was performed to assess the numbers of ticks attached, feeding patterns, and skin erythema, and to collect any detached ticks from the water pan. The numbers of ticks detached and recovered were used to calculate percent recovery and measure the engorgement weights. Erythema at the tick bite sites was assessed by two researchers blinded to the experimental groups and scored based on the percentage of erythematous bite sites on the total of attached ticks.

Infected Tick Challenge and B. burgdorferi Transmission of Guinea Pigs

To generate B. burgdorferi-infected mice and nymphs, B. burgdorferi N40 was inoculated in C3H mice. Approximately, 100 μl of 1×10⁵ N40 spirochetes/ml were injected subcutaneously. I. scapularis larvae were placed on B. burgdorferi-infected C3H mice and fed larvae molted to generate B. burgdorferi-infected nymphs. For Borrelia transmission to guinea pigs, 3 B. burgdorferi N40 infected nymphs were placed on each guinea pig (at least 5 animals in each group) and allowed to feed till the appearance of erythema (up to 120 h post-tick-challenge), after which ticks were pulled-off carefully using forceps. All control and experimental animals were examined for erythema in a double-blinded manner. After tick detachment, the transmission was assessed by culture and by quantitative PCR of skin punches at 3 weeks. Historical studies have examined skin, blood, spleen and bladder after B. burgdorferi-infected ticks were allowed to engorge on guinea pigs and were not able to detect spirochetes tissues other than skin. In several of the animals in the current study, numerous internal sites were examined, and consistent with the previous experiment, B. burgdorferi was detected only in the skin of the guinea pigs. An additional experiment was performed in which one B. burgdorferi-infected tick was placed on each control and experimental animals, and the ticks were allowed to attempt to take a blood meal until they naturally detached from the animals.

### Quantitative PCR to Estimate Spirochete Burden

Guinea pig skin punch biopsies were obtained from sites near and distal to tick attachment sites at 3 weeks, after tick engorgement. The biopsies were suspended in DNAeasy suspension buffer (Qiagen, CA) containing proteinase K and processed for DNA isolation using the DNAeasy kit (Qiagen) according to the manufacturer's protocol. DNA was analyzed by quantitative PCR using the iTaq Sybr Green Supermix (Biorad, CA), for the presence of Borrelia using flaB primers (flaB F-5′ ttcaatcaggtaacggcaca 3′ andflaB R-5′ gacgcttgagaccctgaaag 3′ and results normalized using actin primers (actin F-5′ agcgggaaatcgtgcgtg 3′ and actin R-5′ cagggtacatggtggtgcc 3′).

### Statistical Analysis

Data for ELISA, skin erythema, engorgement weights, and tick detachment/recovery were analyzed by Prism 9.0 software (GraphPad Software, CA). The significance of the difference between controls and experimental groups was analyzed by ordinary ANOVA, two-way ANOVA or Mann-Whitney/Welch's test (as mentioned in respective figure legends). P≤0.05 was considered statistically significant.

### Example 7: Immunization with Plasmid DNA, mRNA or Protein

Guinea pigs exposed to multiple tick infestations develop an adaptive immune response towards tick salivary proteins deposited in the skin during the feeding process. Upon tick challenge in an immune animal, the deposited tick antigens are rapidly recognized, resulting in epicutaneous erythema, severe epidermal hyperplasia, edema and hyperkeratosis at the tick bite site, leading to decreased tick engorgement and rejection. However, the array of protective antigens has yet to be identified. Here, DNA, nucleoside-modified mRNA and protein-based immunization strategies were compared for tick salivary protein 14 (Salp14), an antigen that has been previously demonstrated to be associated with partial tick immunity.

For DNA vaccination, the gene encoding salp14 was cloned into the VR2010 plasmid. Guinea pigs were immunized intradermally with 80 μg of plasmid DNA encoding salp14 or empty vector. Immunized guinea pigs received 2 booster vaccinations every 4 weeks. Similarly, nucleoside-modified mRNA lipid nanoparticles encoding (mRNA-LNPs) salp14 or murineIL-21 (muIL-21, control) were delivered intradermally, with two boosts every 4 weeks (20 μg). muIL-21 has no impact on tick feeding and was therefore used as a negative control. For protein immunizations, guinea pigs received recombinant Salp14 (20 μg), followed by 2 boosts (FIG. 7A). Additionally, the instant inventors performed a slow-delivery immunization 21, in order to more closely mimic a tick bite. For sustained immunization, a total dose of recombinant salp14 (20 μg) was immunized intradermally over the course of one week. Guinea pigs received one booster (single dose) immunization two weeks after the primary immunization.

### Example 8: DNA, mRNA and Protein Immunization Elicit a Comparable Humoral Response

The development of Salp14-specific antibodies was compared among the different immunization strategies. Sera were obtained from each group prior to tick challenge and assessed by ELISA. Salp14 mRNA immunization was the platform that induced the strongest humoral response with Salp14-specific antibodies detected at all dilutions tested (1:500, 1:5,000, 1:50,000 and 1:500,000). Salp14 protein immunization resulted in antibody titers detected up to a dilution of 1:50,000) and salp14 DNA vaccination produced antibody titers evident up to a dilution of (1:5,000). These results indicate that all immunization strategies elicited a humoral response; however, mRNA encoding salp14 generated the most robust response (FIG. 7B).

### Example 9: Erythema at the Bite Site Upon Tick Challenge

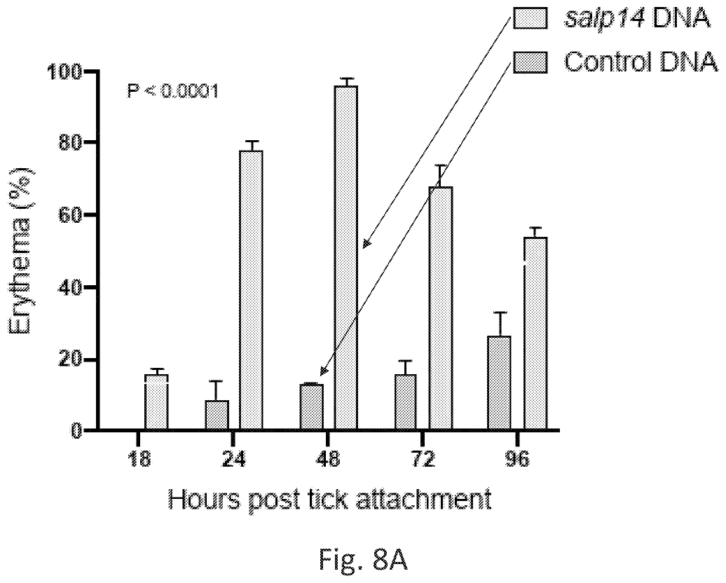

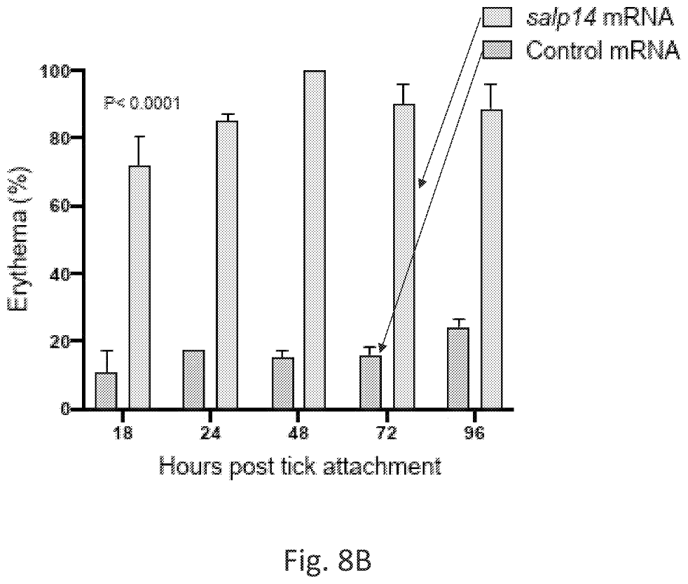

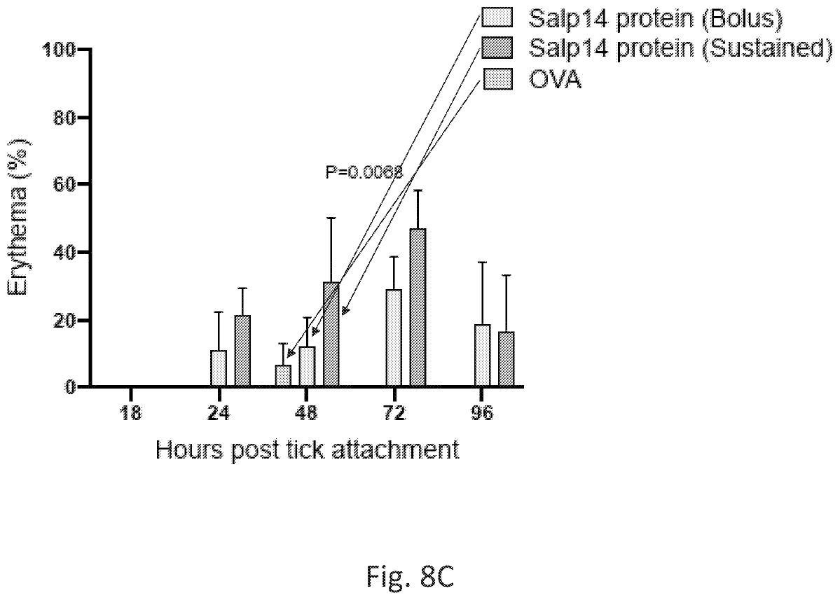

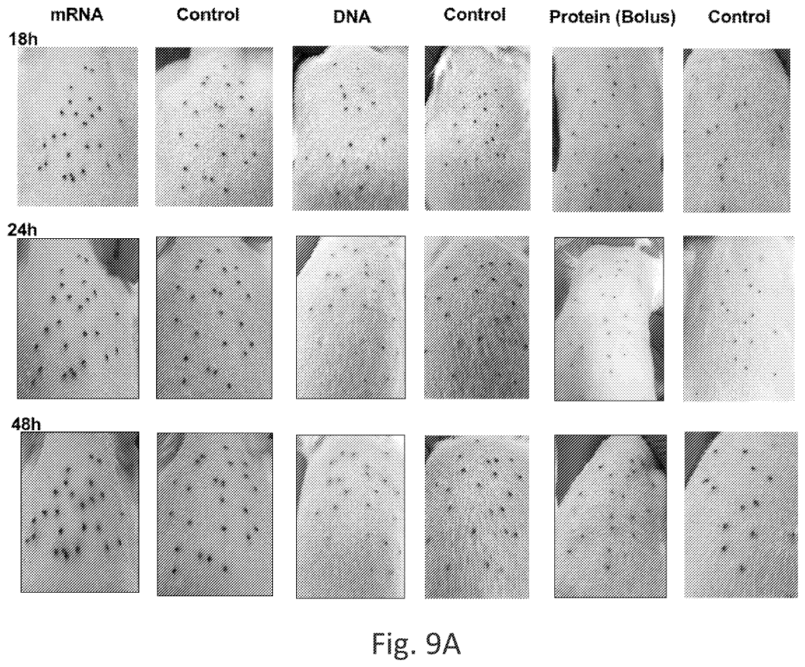

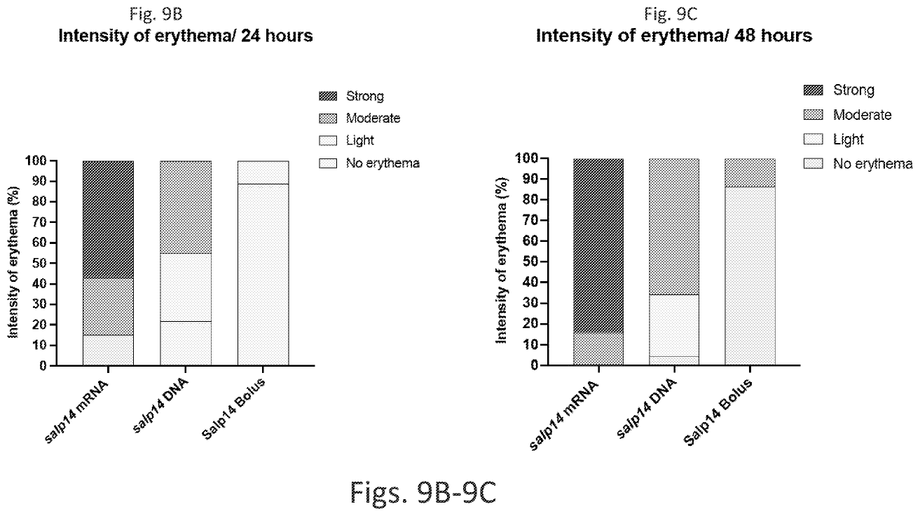

Immunized guinea pigs were challenged with approximately 25 I. scapularis nymphs two weeks after the final boost. Erythema at the bite site is a strong indicator of an immune response to ticks, and one of the initial hallmarks of acquired tick resistance. Guinea pigs were monitored for signs of erythema at the bite site following tick challenge. Erythema was detected in all immunization groups by 24 h after tick attachment (FIGS. 8A-8C and 9A). Guinea pigs immunized with the salp14 mRNA elicited the most robust, and intense erythema at the bite site among all the immunization groups (FIGS. 9B and 9C). Additionally, significant erythema was observed by as early as 18 h in this experimental group, relative to control-immunized guinea pigs and the other platforms (FIGS. 8B and 9A).

### Example 10: Tick Rejection Among Vaccinated Guinea Pigs

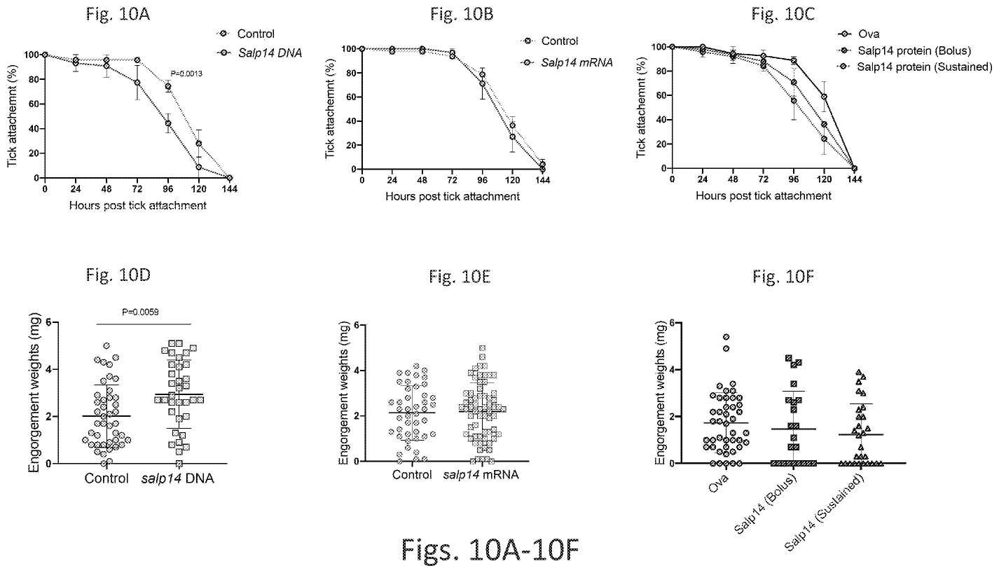

After tick challenge, guinea pigs were monitored daily for evidence of tick rejection, including the rate of tick detachment and engorgement (tick weight) at the bite site. Immunization using a salp14 DNA plasmid resulted in modest tick rejection at day 4 after tick attachment (FIG. 10A). Immunization with the salp14 mRNA did not impact the rate of tick detachment (FIG. 10B). Immunization with Salp14 protein resulted in modest tick rejection at days 4-5 post-tick attachment for both sustained immunizations, as well as bolus immunization (FIG. 10C). Also, sustained immunization resulted in an increase in tick detachment at day 4 (FIG. 10C). Although immunization with mRNA-LNPs did not reject the ticks, immunization resulted in the greatest erythema and dermal inflammation at the bite site, possibly from infiltrating T cells.

As an additional indicator of tick feeding, the engorgement weights of the collected ticks were assessed. Immunization with the DNA plasmid encoding salp14 did not impair engorgement weights relative to control (2.9 mg versus 2.0 mg) (FIG. 10D). Similarly, immunization with the salp14 mRNA did not alter engorgement weights (2.2 mg versus 2.1 mg), nor did immunization with recombinant protein (2.75 mg versus 2.1 mg) (FIG. 10E-10F). Sustained immunization also did not significantly alter engorgement weights (1.2 mg versus 1.7 mg).

### Example 11: Antigenic Regions of Salp14

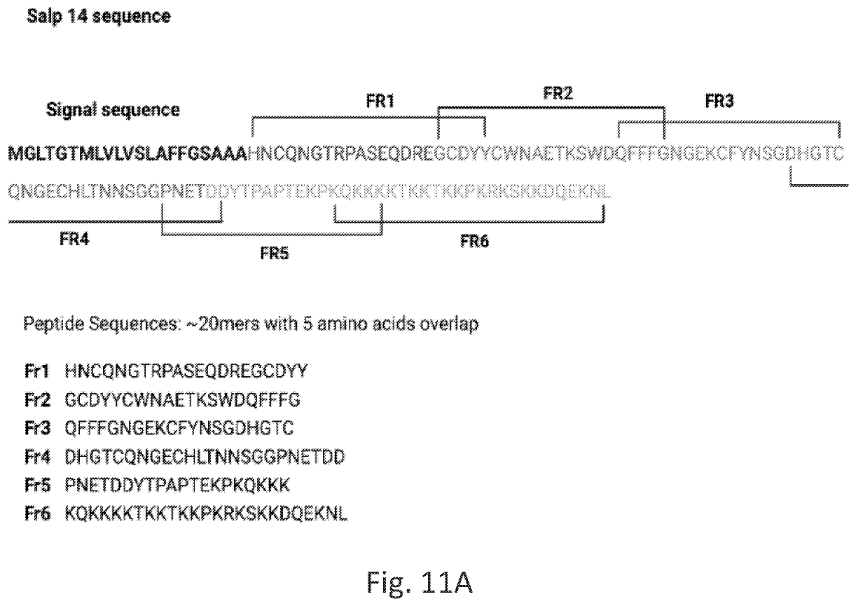

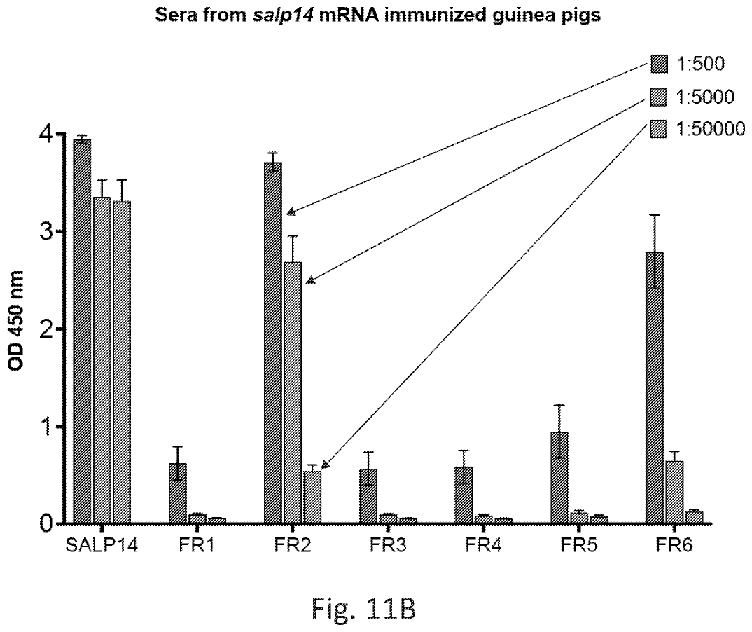

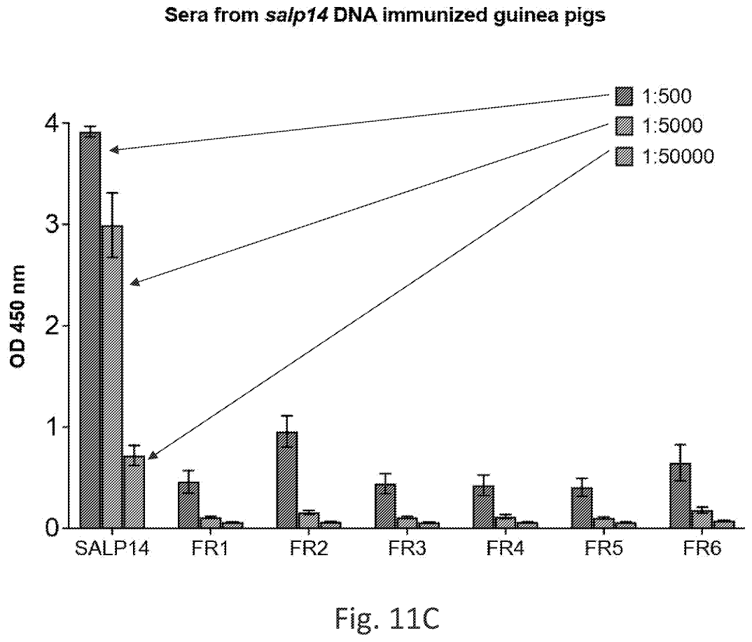

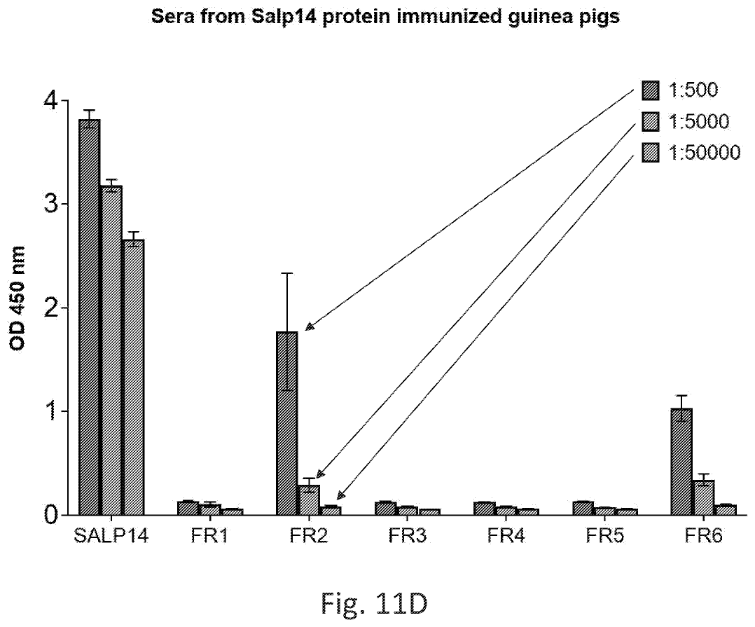

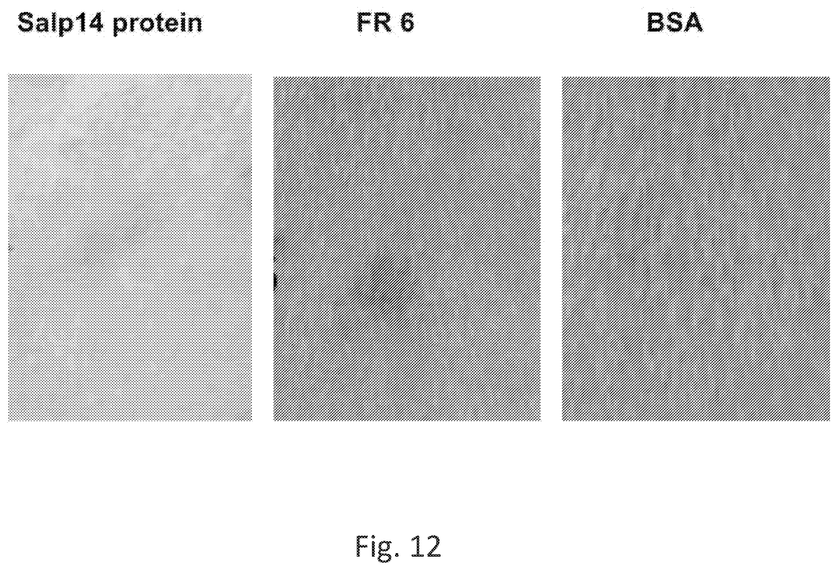

After establishing the antigenicity of Salp14, dominant antigenic epitopes within the salp14 sequence were characterized. The soluble portion of the Salp14 protein (without signal peptide) was divided into six peptides, each approximately 20 amino acid long, with an overlapping sequence of 5 amino acids (fragments, Fr 1-6, FIG. 11A).

Next it was tested which of the six peptides were recognized by antibodies in the sera from guinea pigs immunized with Salp14 protein, salp14 DNA or salp14 mRNA. Interestingly, Fr 2 and Fr 6 were recognized by antibodies in the sera of salp14-mRNA-immunized guinea pigs with the highest titers (FIG. 11B). In comparison, sera from Salp14 protein-immunized animals and salp14-DNA-immunized guinea pigs showed weaker responses to these peptides, consistent with their lower level of general antibodies against salp14 antibodies (FIGS. 11B-11D). No other peptides were readily recognized by these sera. This indicates that Fr 2 and Fr 6 are the predominant regions of salp14 recognized by antibodies that recognize linear epitopes using all of the regimens examined. Conformational epitopes bound by antibodies, which could involve other regions of salp14, were not mapped.

To further test the immunoreactivity of salp14 peptides in vivo, tick immune guinea pigs, which had been repeatedly infested with I. scapularis, were utilized. Skin testing was performed by injecting 2 μg of peptide intradermally on these guinea pigs, in order to evaluate immune reactivity that is associated with local erythema. Salp14 protein was taken as a positive control and BSA was taken as negative control. As shown in FIG. 12, Salp14 and Fr 6 showed an evident of an inflammatory dermal response, lasting for at least 72 hours. No other peptide or BSA showed any skin response. These results further demonstrate that the C-terminal region of Salp14 is important in the genesis of immunoreactivity associated with early skin immune response to tick bites, a signature for the initiation of acquired tick resistance in guinea pigs.

### Example 12: Selected Discussion

Evidence supporting the development of an effective anti-tick vaccine are derived from experiments demonstrating that laboratory animals, such as rabbits and guinea pigs, can acquire resistance to tick feeding following multiple tick infestations, referred to as “tick immunity.” Importantly, guinea pigs with tick immunity to I. scapularis can be protected from infection by tick-borne B. burgdorferi. Guinea pigs exposed to multiple tick infestations develop an adaptive immune response towards tick salivary proteins deposited in the skin during the feeding process. Upon tick challenge in an immune animal, the deposited tick antigens are rapidly recognized, resulting in epicutaneous erythema, epidermal hyperplasia, edema, and hyperkeratosis at the bite site, leading to decreased tick engorgement and rejection. Importantly, immunity to I. scapularis can also influence the ability of Amblyomma americanum and Dermacentor andersonii to successfully feed on a host.

While antigen discovery is important, vaccine delivery is also critical in eliciting protective immunity. To date, immunization with a single or cocktail of tick salivary proteins not yet reproduced tick immunity equivalent to naturally acquired tick resistance. Identifying vaccine delivery protocols can be an important factor in how vaccine candidates are screened. A non-limiting goal of this study is to address limitations in the understanding of vaccine delivery against tick salivary proteins. Salp14 was used to compare different immunization platforms in eliciting some of the markers of tick immunity, including erythema, decreased tick attachment and engorgement. This study demonstrates that guinea pigs immunized with salp14 nucleoside-modified mRNA-LNP developed substantial erythema at the tick bite site within the first 18 hours. Guinea pigs immunized with salp14 DNA or Salp14 protein started to develop erythema only after 24 h. In addition, the erythema was not as pronounced as the salp14 mRNA-LNP vaccinations. Erythema is one of the parameters associated with tick immunity, with the potential to generate itching and pain. Histological examination of the tick bite-sites on tick-immune animals, reveals that erythema is the result of degranulation of immune cells such as eosinophils, mast cells, and basophils migrating to the bite site.

While identifying the antigenic region of Salp14, guinea pigs immunized with salp14 mRNA, salp14 DNA or recombinant protein, all elicited antibody responses, primarily by fragments 2 and 6, with 6 comprising part of C terminus of Salp14. Furthermore, skin testing of the various Salp14 epitopes demonstrated that only fragment 6 elicited erythema, when injected into the dermis of guinea pigs that were naturally made tick immune by repeated tick infestation. Collectively, these studies indicate that the C terminal region of Salp14 is associated with responses, likely humoral and/or cellular, linked to tick immunity.

Although evidence for tick immunity in humans has been difficult to study in large controlled studies, there are reported cases of individuals developing hypersensitivity reactions at the tick bite site, similar to the observation in tick immune animals. Additionally, individuals with frequent exposures to tick bites have been shown to develop antibodies to tick proteins, confirming observations in laboratory tick immunity models. Importantly, individuals that report itching at the tick bite site have a decreased probability of acquiring B. burgdorferi. These results indicate that previous exposure to ticks can induce protective immunity, resulting in erythema at the bite site upon future bites. This is a strategy to prevent transmission of B. burgdorferi since transmission is not known to occur during the first 24 hours of tick attachment. Indeed, if a human noticed a tick bite due to increased redness and/or itching, the immediate response would be to remove the tick, and this would likely result in protection from infection with the Lyme disease agent. In contrast, later phenomenon that are associated with tick immunity, such as decreased attachment and altered engorgement may not be as critical as early recognition and removal of the tick for the prevention of human Lyme disease. Certainly, erythema would be evident only on areas of skin that are observable by an individual, but this comprises a substantial portion of the body. In addition, if pruritis occurred in unobserved area of the skin, this would still result in identification of the tick, and early removal.

Overall, the results of this study compare immunization strategies that are best able to elicit the first main component of tick immunity—early erythema at the tick bite site. Nucleoside-modified mRNA-LNP salp14 immunization elicits a greater degree of redness, and earlier erythema than either DNA immunization or protein immunization. The method of immunization, as well as the selection of antigens must both be considered, as anti-tick vaccines continue to be developed to help aid humans in the prevention of Lyme disease, and possibly other tick-borne infections.

### Example 13: Materials and Methods for Examples 7-11

### Ticks and Experimental Animals

I. scapularis nymphs were obtained from Oklahoma State University (Stillwater, OK, USA). Ticks were kept in the incubator at 23° C. and 85% relative humidity under a 14 h light, 10 h dark photoperiod at the Infectious Diseases Laboratory, Department of Internal Medicine, Yale University School of Medicine. 4-5-weeks old female Hartley guinea pigs (Charles River laboratory, MA) were used for immunizations and for tick challenge experiments.

Formulation of Salp14 mRNA-LNP, Plasmid DNA and Recombinant Protein

mRNA-LNP-Salp 14 (accession number AF209921) and muIL-21 mRNA-LNPs were generated as previously described by Freyn, A. W. et al., Mol Ther 28, 1569-1584, doi:10.1016/j.ymthe.2020.04.018 (2020). Briefly, mRNAs were transcribed to contain 101 nucleotide-long poly(A) tails. N-1-methylpseudouridine (m1Ψ-5′)-triphosphate (TriLink) instead of UTP was used to generate modified nucleoside-containing mRNA. Capping of the in vitro transcribed mRNAs was performed co-transcriptionally using the trinucleotide cap1 analog, CleanCap (TriLink). mRNA was purified by cellulose purification, as described in Baiersdorfer, M et al. Mol Ther Nucleic Acids 15, 26-35, doi:10.1016/j.omtn.2019.02.018 (2019) (incorporated herein by reference). All mRNAs were analyzed by agarose gel electrophoresis and were stored frozen at −20° C. The mRNA was then encapsulated in LNPs using an aqueous solution of mRNA at acidic pH 4.0 and mixed with a solution of lipids (Maier, M. A. et al. Mol Ther 21, 1570-1578, doi:10.1038/mt.2013.124 (2013) and Jayaraman, M. et al. Angew Chem Int Ed Engl 51, 8529-8533, doi:10.1002/anie.201203263 (2012), both of which are hereby incorporated by reference), consisting of an ionizable cationic lipid/phosphatidylcholine/cholesterol/PEG-lipid (proprietary of Acuitas, Vancouver, Canada) (50:10:38.5:1.5 mol/mol). For encapsulation, RNA was mixed with the lipids at al ratio of ˜0.05 (wt/wt). The LNP had a diameter of ˜80 nm as measured by dynamic light scattering using a Zetasizer Nano ZS (Malvern Instruments Ltd, Malvern, UK) instrument, and stored at −80° C.

DNA—salp14 gene was PCR amplified using I. scapularis salivary gland cDNA (described below) and cloned into the VR2010 vector derived from VR1020 plasmid (Vical, Inc.). The cloning was performed under the restriction sites BamHI and BglII using Gibson assembly cloning kit (New England Biolabs) and the sequence of clone was validated by DNA sequencing (Keck sequencing facility, Yale University). For immunization, plasmids were purified using Endo-free plasmid purification kit (Qiagen), as previously described.

Recombinant protein—Total RNA was isolated from the salivary glands of fed I. scapularis ticks using Trizol (Life Technologies, Inc) and cDNA was synthesized according to the manufacture's protocol (iScript cDNA synthesis kit, Bio-RAD). Gene-specific primers were used to amplify the salp14 and the amplicon was cloned into pMT-Bip-V5-HisA vector. To validate the clones, the recombinant DNA was sequenced at the Keck sequencing facility, Yale University. Recombinant Salp 14 protein was generated using the Drosophila expression system according to the manufacturer's protocol (Invitrogen, CA) and as described for tick salivary proteins elsewhere (Narasimhan, S. et al., Ticks Tick Borne Dis 11, 101369, doi:10.1016/j.ttbdis.2019.101369 (2020), Anguita, J. et al. Immunity 16, 849-859, doi:10.1016/s1074-7613(02)00325-4 (2002), and Murfin, K. E. et al., Ticks Tick Borne Dis 10, 1124-1134, doi:10.1016/j.ttbdis.2019.06.002 (2019), all incorporated herein by reference). The purity of the recombinant protein was assessed by SDS-PAGE using 4-20% gradient precast gels (Biorad, CA) and quantified using the BCA protein assay kit (Thermo Scientific, MA).

### Guinea Pig Immunization

Female guinea pigs (4-5-weeks old) were immunized intradermally with 20 μg of Salp14 mRNA-LNPs, or human (hu)IL-21 mRNA-LNP (control), 80 μg of plasmid DNA encoding Salp14 or empty plasmid constructions VR2010, 20 μg of recombinant Salp14 or Ovalbumin (OVA control) and sustained immunization with 20 μg of recombinant Salp14 over the course of one week.

The animals were boosted twice at 4-week intervals. Two weeks after the last boost, the animals were bled retro-orbitally to obtain 500 μl of blood and the serum separated for use in ELISA experiments. At least 3 animals were used in each group.

### Tick Challenge

Prior to tick challenge, the backs of guinea pigs were carefully shaved. Guinea pigs were anesthetized with a mixture of 40 mg/kg ketamine/xylazine and 25-30 I. scapularis nymphs were applied to the shaved backs. Guinea pigs were housed individually in cages with wire racks above water to recover engorged and/or rejected ticks. Animals were monitored multiple times throughout the day for evidence of tick rejection.

Visual inspection was performed to determine the degree of erythema at the site of the tick bite at 24 and 48 hours. Erythema was scored as: No erythema (−), Light (+), Moderate (++) and Strong (+++). The percentage of the degree of erythema was generated from these data and is represented in FIGS. 9B and 9C.

### Generation of Salp14 Peptides and Skin Testing

The sequence of Salp14 protein (104 aa without signal peptide) was divided into 6 overlapping peptides, each ˜20 aa long, as follows: HNCQNGTRPASEQDREGCDYY (Fr1), GCDYYCWNAETKSWDQFFFG (Fr2), QFFFGNGEKCFYNSGDHGTC (Fr3), DHGTCQNGECHLTNNSGGPNETDD (Fr4), PNETDDYTPAPTEKPKQKKK (Fr5) and KQKKKKTKKTKKPKRKSKKDQEKNL (Fr6). The peptides were synthesized by GenScript (Piscataway, NJ).

Guinea pigs were made tick-immune by being fed upon with I. scapularis ticks for four days, twice, at an interval of two weeks. For skin testing, 2 μg of peptides, recombinant Salp14 (positive control) or bovine serum albumin-BSA (negative control) were injected intradermally, on the saved backs of tick-immune guinea pigs. The animals were monitored for the generation of skin redness at the site of injection for 96 hours.

### ELISA Assessment

Sera were obtained from the blood of guinea pigs collected by retro-orbital bleeds. To determine antigen-specific antibody responses, ELISAs were performed. Briefly, 96-well plates were coated overnight with the indicated proteins and peptides (250 ng) diluted in carbonate-bicarbonate buffer, pH 9.6. The wells were then washed with PBST (PBS containing 0.05% Tween-20) and blocked using PBS supplemented with 3% BSA. Sera were serially diluted and incubated for 2 hours at 37° C. The wells were the washed and incubated with the secondary goat anti-guinea pig IgG-HRP antibody (ThermoFisher, Waltham, A, USA), After washing, TMB HRP substrate solution was added and incubated for 30 min, followed by addition of TMB stop solution. The plates were read at 450 nm.

### Statistical Analysis

Statistical analysis was performed using Prism 8.0 software (GraphPad Software, CA). The numbers of animals used in each experiment are indicated in the figure legend. Statistical significance was determined using two-way ANOVA, two-tailed student t-test and Mann Whitney test.

### Enumerated Embodiments

In some embodiments, the present invention is directed to the following non-limiting embodiments:

Embodiment 1: A method of generating tick immunity in a subject, the method comprising administering to the subject in need thereof a therapeutically effective amount of at least one tick-salivary protein, wherein the at least one tick-salivary protein comprises at least one protein selected from the group consisting of: Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09.

Embodiment 2: The method of Embodiment 1, wherein the at least one tick-salivary protein comprises Salp14, Salp26A, TSLPI, IsPDIA3, TIX5, P32, and SG27.

Embodiment 3: The method of Embodiment 2, wherein the at least one tick-salivary protein further comprises at least one protein selected from the group consisting of Salp10, Salp15, Salp25A, Salp25B, Salp25C, Salp25D, P11, Salp16A, Salp17, Salp12, SG10, and SG09.

Embodiment 4: The method of Embodiment 1, wherein the at least one tick-salivary protein comprises Salp14, Salp15, Salp25D, Salp26A, TSLPI, IsPDIA3, TIX5, P32, SG10, and SG27.

Embodiment 5: The method of Embodiment 1, wherein the at least one tick-salivary protein comprises Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09

Embodiment 6: The method of Embodiment 1, further comprising administering an adjuvant to the subject.

Embodiment 7: The method of Embodiment 6, wherein the adjuvant is at least one selected from the group consisting of incomplete Freund's adjuvant, alum, addavax (equivalent to MF59), MF59, and AS03.

Embodiment 8: The method of Embodiment 1, wherein the at least one tick-salivary protein is administered by at least one route selected from the group consisting of inhalational, oral, rectal, vaginal, parenteral, intracranial, topical, transdermal, intradermal, subcutaneous, pulmonary, intranasal, buccal, ophthalmic, intrathecal, and intravenous.

Embodiment 9: The method of Embodiment 1, wherein the subject is a mammal.

Embodiment 10: The method of Embodiment 9, wherein the subject is a human.

Embodiment 11: A composition comprising a therapeutically effective amount of at least one tick-salivary protein, wherein the at least one tick-salivary protein comprises at least one protein selected from the group consisting of: Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09.

Embodiment 12: The composition of Embodiment 11, wherein the at least one tick-salivary protein comprises Salp14, Salp26A, TSLPI, IsPDIA3, TIX5, P32, and SG27.

Embodiment 13: The composition of Embodiment 12, wherein the at least one tick-salivary protein further comprises at least one protein selected from the group consisting of Salp10, Salp15, Salp25A, Salp25B, Salp25C, Salp25D, P11, Salp16A, Salp17, Salp12, SG10, and SG09.

Embodiment 14: The composition of Embodiment 11, wherein the at least one tick-salivary protein comprises Salp14, Salp15, Salp25D, Salp26A, TSLPI, IsPDIA3, TIX5, P32, SG10, and SG27.

Embodiment 15: The composition of Embodiment 11, wherein the at least one tick-salivary protein comprises Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09.

Embodiment 16: The composition of Embodiment 11, further comprising an adjuvant.

Embodiment 17: The composition of Embodiment 16, wherein the adjuvant is at least one selected from the group consisting of incomplete Freund's adjuvant, alum, addavax (equivalent to MF59), MF59, and AS03.

Embodiment 18: The composition of Embodiment 11, wherein the composition further comprises at least one pharmaceutically acceptable carrier.

Embodiment 19: The composition of Embodiment 11, wherein the composition is formulated for administration by at least one route selected from the group consisting of inhalational, oral, rectal, vaginal, parenteral, intracranial, topical, transdermal, pulmonary, intranasal, buccal, ophthalmic, intrathecal, and intravenous.

Embodiment 20: The composition of Embodiment 11, wherein the composition is a pharmaceutical composition for generating tick immunity in a subject.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this specification has been disclosed with reference to specific embodiments, it is apparent that other embodiments and variations of this specification may be devised by others skilled in the art without departing from the true spirit and scope of the specification. The appended claims are intended to be construed to include all such embodiments and equivalent variations.

What is claimed is:

1. A method of generating tick immunity in a subject, the method comprising administering to the subject in need thereof a therapeutically effective amount of at least one tick-salivary protein, wherein the at least one tick-salivary protein comprises at least one protein selected from the group consisting of: Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09.

2. The method of claim 1, wherein the at least one tick-salivary protein comprises Salp14, Salp26A, TSLPI, IsPDIA3, TIX5, P32, and SG27.

3. The method of claim 2, wherein the at least one tick-salivary protein further comprises at least one protein selected from the group consisting of Salp10, Salp15, Salp25A, Salp25B, Salp25C, Salp25D, P11, Salp16A, Salp17, Salp12, SG10, and SG09.

4. The method of claim 1, wherein the at least one tick-salivary protein comprises Salp14, Salp15, Salp25D, Salp26A, TSLPI, IsPDIA3, TIX5, P32, SG10, and SG27.

5. The method of claim 1, wherein the at least one tick-salivary protein comprises Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09

6. The method of claim 1, further comprising administering an adjuvant to the subject.

7. The method of claim 6, wherein the adjuvant is at least one selected from the group consisting of incomplete Freund's adjuvant, alum, addavax (equivalent to MF59), MF59, and AS03.

8. The method of claim 1, wherein the at least one tick-salivary protein is administered by at least one route selected from the group consisting of inhalational, oral, rectal, vaginal, parenteral, intracranial, topical, transdermal, intradermal, subcutaneous, pulmonary, intranasal, buccal, ophthalmic, intrathecal, and intravenous.

9. The method of claim 1, wherein the subject is a mammal.

10. The method of claim 9, wherein the subject is a human.

11. A composition comprising a therapeutically effective amount of at least one tick-salivary protein, wherein the at least one tick-salivary protein comprises at least one protein selected from the group consisting of: Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09.

12. The composition of claim 11, wherein the at least one tick-salivary protein comprises Salp14, Salp26A, TSLPI, IsPDIA3, TIX5, P32, and SG27.

13. The composition of claim 12, wherein the at least one tick-salivary protein further comprises at least one protein selected from the group consisting of Salp10, Salp15, Salp25A, Salp25B, Salp25C, Salp25D, P11, Salp16A, Salp17, Salp12, SG10, and SG09.

14. The composition of claim 11, wherein the at least one tick-salivary protein comprises Salp14, Salp15, Salp25D, Salp26A, TSLPI, IsPDIA3, TIX5, P32, SG10, and SG27.

15. The composition of claim 11, wherein the at least one tick-salivary protein comprises Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, and SG09.

16. The composition of claim 11, further comprising an adjuvant.

17. The composition of claim 16, wherein the adjuvant is at least one selected from the group consisting of incomplete Freund's adjuvant, alum, addavax (equivalent to MF59), MF59, and AS03.

18. The composition of claim 11, wherein the composition further comprises at least one pharmaceutically acceptable carrier.

19. The composition of claim 11, wherein the composition is formulated for administration by at least one route selected from the group consisting of inhalational, oral, rectal, vaginal, parenteral, intracranial, topical, transdermal, pulmonary, intranasal, buccal, ophthalmic, intrathecal, and intravenous.

20. The composition of claim 11, wherein the composition is a pharmaceutical composition for generating tick immunity in a subject.
